(19) United States
(12) Patent Application Publication
(10) Pub. No.: US 2011/0250582 A1
(43) Pub. Date: Oct. 13, 2011

(54) METHODS AND SYSTEMS FOR GAS PRODUCTION FROM A RESERVOIR

(76) Inventors: Ian Donald Gates, Calgary (CA); Jennifer Jane Adams, Calgary (CA); Stephen Richard Larter, Calgary (CA)

(21) Appl. No.: 13/056,874

(22) PCT Filed: Jul. 31, 2009
(86) PCT No.: PCT/CA2009/001069

§ 371 Date: Jun. 24, 2011

(30) Foreign Application Priority Data
Aug. 1, 2008 (CA) 2638451

(51) Int. Cl. C12Q 3/00; C12M 1/36; C12M 1/34

(52) U.S. Cl. 435/3; 435/287.1; 435/286.1

(57) ABSTRACT

A method and system for production of biogenerated gas from a zone in a reservoir are described. Microbial conversion of petroleum in the zone to biogenerated gas is stimulated. The biogenerated gas can be, for example, methane, hydrogen or carbon dioxide, or another gas. Gas including the biogenerated gas is produced from the zone to surface. While producing the gas, production parameters are monitored including reservoir pressure and an isotopic composition of the produced gas. Based on the monitored production parameters, at least one of stimulation of the microbial conversion to control the composition of the produced gas or gas saturation in the zone to control mobility of the biogenerated methane is adjusted.

### TECHNICAL FIELD

This invention relates to gas production.

### BACKGROUND

After primary, secondary and enhanced oil recovery techniques have been employed to recover petroleum (i.e., oil, heavy oil, bitumen and/or natural gas, commonly also termed “hydrocarbons” by the petroleum industry) from subsurface reservoirs, significant amounts of hydrocarbons still can remain unrecoverable in the reservoirs. The recovery of liquid hydrocarbons is typically less than 50% of the original amount of hydrocarbons in place in a reservoir. In heavy oil reservoirs, the recovery can be less than 30%.

With sufficient nutrients, temperature and pressure conditions, over a long geological time period, liquid hydrocarbons can be degraded in situ by anaerobic indigenous microbes. The anaerobic oil biodegradation process converts conventional light oil into methane, carbon dioxide and a petroleum residue, which forms heavy oil or bitumen. This process of methane generation (or methanogenesis) and heavy oil generation, called methanogenic hydrocarbon biodegradation, occurs over millions to tens of millions of years.

It has been recognized that the naturally slow methanogenic biodegradation process can be accelerated to enhance methane production in the reservoir, in hopes that energy can be recovered from previously unrecoverable petroleum (e.g., from heavy oilfields) as methane rather than oil. For example, as described in WO 2005/115649 entitled “Process for Stimulating Production of Methane from Petroleum in Subterranean Formations”, techniques are described for injecting one or more agents into a reservoir in which methanogenic microorganisms are present to modify the reservoir environment to promote in situ microbial degradation of petroleum, promote microbial generation of methane and to demote in situ microbial degradation of methane.

### SUMMARY

This invention relates to gas production. In general, in one aspect, the invention features a method for producing biogenerated gas from a zone in a reservoir. Microbial conversion of petroleum in the zone to biogenerated gas is stimulated. The biogenerated gas can be, for example, methane, hydrogen or carbon dioxide, or another gas.

Gas including the biogenerated gas is produced from the zone to surface. While producing the gas, production parameters are monitored including reservoir pressure and an isotopic composition of at least one of either methane or carbon dioxide or hydrogen included in the produced gas. Based on the monitored production parameters, at least one of stimulation of the microbial conversion to control the composition of the produced gas or gas saturation in the zone to control mobility of the biogenerated methane is adjusted.

Implementations of the invention can include one or more of the following features. Monitoring production parameters can further include monitoring a flow rate of the produced gas, water and/or oil. Monitoring can further include monitoring the gas composition of the produced gas, for example, the amounts of methane, carbon dioxide and/or hydrogen. Monitoring an isotopic composition can include monitoring a carbon isotopic composition of at least one of either methane or carbon dioxide or a hydrogen isotopic composition of methane or hydrogen included in the produced gas. Monitoring an isotopic composition can include monitoring an oxygen isotopic composition of carbon dioxide included in the produced gas. Monitoring production parameters can further include monitoring at least one of either hydrogen or oxygen isotopic composition of produced water or the carbon or hydrogen isotopic composition of produced oil components.

Before producing gas from the zone, an increase in reservoir pressure in the zone can be monitored and, based on the reservoir pressure, the zone's gas saturation can be determined. Production of gas from the zone can be commenced when the zone's gas saturation reaches a threshold gas saturation. The threshold gas saturation is based on a critical gas saturation of the zone. In some implementations, the threshold gas saturation is in the range of approximately 85% to 95% of the critical gas saturation. In other implementations, the threshold gas saturation is the critical gas saturation.

Stimulating microbial conversion of petroleum in the zone to biogenerated gas can include injecting one or more modifiers into the reservoir to stimulate microbial conversion of the petroleum to gas. Fluid can be injected into the reservoir, the fluid including gas and water. Adjusting at least one of stimulation of the microbial conversion to control the composition of the produced gas or gas saturation in the zone to control mobility of the biogenerated methane can include adjusting one or more of the following: an injector well flow rate for injecting at least one of either the one or more modifiers or the fluid into the reservoir; a production well flow rate; a composition of the one or more modifiers injected into the reservoir; a quantity of the one or more modifiers injected into the reservoir; a composition of the one or more modifiers injected into the reservoir; a composition of the gas included in the fluid injected into the reservoir; a quantity of the gas included in the fluid injected into the reservoir; a quantity of the water included in the fluid injected into the reservoir; or a duration of injection, soak and production cycles for the reservoir. Adjusting at least one of stimulation of the microbial conversion to control the composition of the produced gas or gas saturation in the zone to control mobility of the biogenerated methane can include ceasing production of the gas from one or more wells and commencing production of the gas from one or more different wells.

Injecting fluid into the zone can include injecting fluid into the zone through a first set of one or more wells, and producing gas from the zone can include producing gas from the zone through a second set of one or more wells. Injecting fluid into the zone can be concurrent with producing gas from the zone or can cease while producing gas from the zone. Injecting fluid into the zone can include injecting fluid into the zone through a first set of one or more wells and producing gas from the zone can include producing gas from the zone through a second set of one or more wells. A soak cycle can be allowed to endure in a region of the zone situated beneath a third set of one or more wells, while injection and production occur from the first and second set of one or more wells. During a later cycle, the first set of one or more wells can be allowed to endure a soak cycle, fluid can be injected into the zone through the second set of one or more wells and gas can be produced from the zone from the third set of one or more wells.

An isotope fractionation model can be used to predict the isotopic composition of the produced gas when gas production is occurring under optimal conditions. The actual isotopic composition of the produced gas can be compared to the predicted isotopic composition. The comparison can be used to adjust at least one of stimulation of the microbial conversion to control the composition of the produced gas or gas saturation in the zone to control mobility of the biogenerated methane.

In general, in another aspect, the invention features a method for producing biogenerated methane from a zone in a reservoir that can support biodegradation of petroleum to produce biogenerated methane by methanogenesis. One or more modifiers are injected into the reservoir to stimulate microbial conversion of the petroleum to biogenerated methane. Fluid is injected into the reservoir, the fluid including gas and water. While gas saturation in the zone exceeds a critical gas saturation of the zone, gas is produced from the zone to surface including producing the biogenerated methane. While producing the gas, production parameters are monitored including the pressure in the reservoir, gas production flow rate and composition of the produced gas, including monitoring an isotopic composition of at least one of either methane or carbon dioxide or hydrogen included in the produced gas. Based on the monitored production parameters, injection into and/or production from the zone are controlled to enhance production of biogenerated methane from the zone.

Implementations of the invention can include one or more of the following features. Controlling injection into and/or production from the zone to enhance production of biogenerated methane from the zone can include controlling one or more of the following: an injector well flow rate; a production well flow rate; a composition of the one or more modifiers injected into the reservoir; a quantity of the one or more modifiers injected into the reservoir; a composition of the gas injected into the reservoir; a quantity of the gas injected into the reservoir; or a duration of injection, soak and production cycles for the reservoir.

Before producing gas from the zone, an increase in reservoir pressure in the zone in response to an increase in biogenerated methane and the injected fluid can be monitored. Based on the reservoir pressure, the zone's gas saturation can be determined. Production of gas from the zone can be commenced when the zone's gas saturation reaches a threshold gas saturation. The threshold gas saturation is based on the critical gas saturation. In some implementations, the threshold gas saturation is in the range of approximately 85% to 95% of the critical gas saturation. In other implementations, the threshold gas saturation is the critical gas saturation.

An isotope fractionation model can be used to predict the composition of the produced gas when gas production is occurring under optimal conditions. The actual composition of the produced gas can be compared to the predicted composition. The comparison can be used to control injection into and/or production from the zone to enhance production of biogenerated methane from the zone.

In general, in another aspect, the invention features a system for producing biogenerated gas from a zone in a reservoir. The system includes an isotope fractionation model simulator configured to predict a composition of produced gas from the zone when production of biogenerated gas is occurring under optimal conditions. The system further includes a production parameter monitoring engine configured to receive production parameter data about a production process, the production parameter data including reservoir pressure and an isotopic composition of at least one or either methane or carbon dioxide or hydrogen included in the produced gas. The system further includes an analysis engine configured to receive the predicted compositions of produced gas from the isotope fractional model simulator and the production parameter data from the production parameter monitoring engine. The analysis engine is further configured to compare the predicted composition of produced gas to the actual isotopic composition of the produced gas and provide an analysis of the efficacy of the production process based on the comparison.

Implementations of the invention can include one or more of the following features. The production parameters monitoring engine can be further configured to receive production parameter data including a bulk gas composition of the produced gas. The analysis engine can be further configured to provide control signals to a controller for the production process to adjust the production process in response to the analysis. The analysis engine is further configured to provide control signals to a controller for an injection process injecting at least one of modifiers to stimulate microbial conversion of petroleum to biogenerated gas or fluid including gas and water into the reservoir, where the control signals adjust the injection process in response to the analysis.

Implementations of the invention can realize one or more of the following advantages. Unrecoverable oil can be recovered as a biogenerated gas, for example, as methane. A reservoir can be re-pressurized, which can facilitate subsequent oil recovery operations Enhanced gas production from an oil reservoir can be achieved. Energy can be recovered from a reservoir in a lower carbon content form (e.g., as methane (CH₄)) than if recovering heavy oil or bitumen (approximately CH_{1.5}). Fuel can be produced from a reservoir in a form that does not require upgrading, for example, as compared to heavy oil. Energy and environmental impacts can be reduced when recovering fuel from heavy oil and bitumen reservoirs as a biogenerated gas rather than as heavy oil or bitumen. Additional economic gains can be realized from a non-productive oil reservoir by converting and then producing hydrocarbons therein as gas.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a flowchart showing an example process for enhancing production of biogenerated gas.

FIG. 2 is a schematic representation of a cross-sectional view of two horizontal wells in a reservoir.

FIG. 3 is a graph of cumulative gas produced versus time in an example with no gas injection.

FIG. 4 is a graph of cumulative gas produced versus time in an example with gas injection.

FIG. 5 is a schematic representation of a cross-sectional view of horizontal well in a reservoir.

FIG. 6 is a graph of cumulative gas produced versus time.

FIG. 7 is a graph of well bottom-hole pressure versus time.

FIG. 8 is a graph of well bottom-hole pressure version time.

FIG. 9 is a schematic representation of a five-spot well pattern arrangement.

FIG. 10 is a graph showing injection pressures of individual wells arranged in the five-spot pattern of FIG. 9.

FIG. 11 is a graph showing fluid injection rates of individual wells arranged in the five-spot pattern of FIG. 9.

FIG. 12 is a graph showing cumulative gas volumes produced over time from the individual wells arranged in the five-spot pattern of FIG. 9.

FIG. 13 is a schematic representation of an example well arrangement in a multiple well operation.

FIG. 14 is a flowchart showing an example process using the Rayleigh isotope fractionation model to assess the process of methanogenic biodegradation.

FIG. 15 is a schematic representation of the Rayleigh isotope fractionation model.

FIGS. 16A-D are example simulation plots based on an isotope fractionation model.

FIG. 17 is a graph showing the mole % of carbon dioxide in produced gas versus the carbon isotopic composition of produced methane.

FIG. 18 is a graph showing the mole % of carbon dioxide in produced gas versus the carbon isotopic composition of produced carbon dioxide.

FIG. 19 is a schematic representation of an example system that can be used to enhance production of biogenerated gas from a zone in a reservoir.

FIG. 20 is a schematic representation of an example computer system.

Like reference symbols in the various drawings indicate like elements.

### DETAILED DESCRIPTION

Methods and systems for producing biogenerated gas from a reservoir are described. Biogenerated gas can include any gas produced in situ in a reservoir by biodegradation. For illustrative purposes, the methods and systems are described herein in the context of producing biogenerated methane. However, it should be understood that the methods and systems can apply to other biogenerated gases, e.g., hydrogen, carbon dioxide, and that methane is but one example.

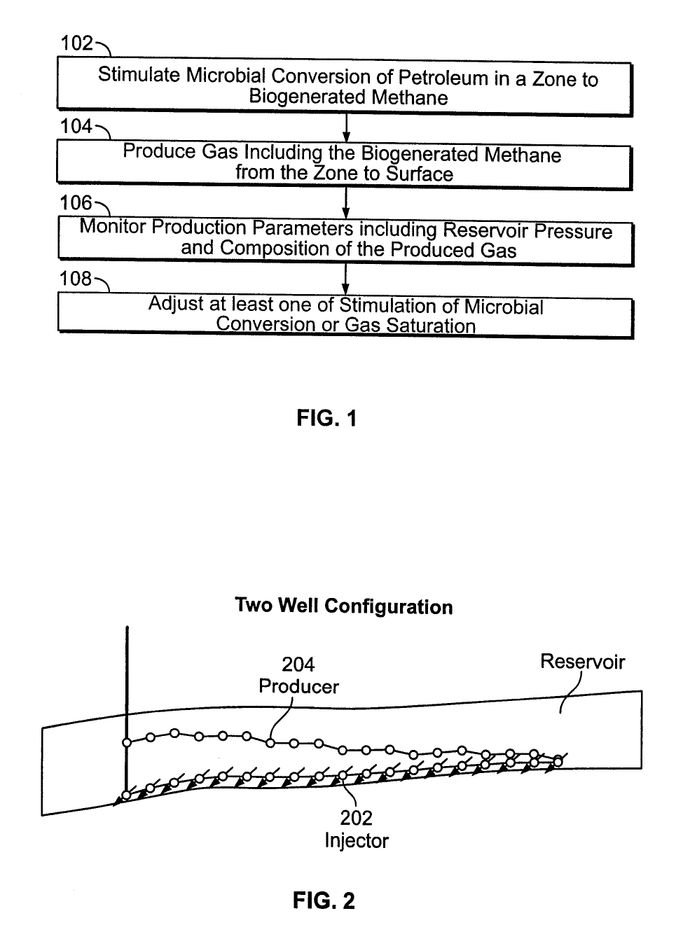

Referring to FIG. 1, a flowchart shows an example process 100 for producing biogenerated gas from a reservoir. Microbial conversion of petroleum to methane in a zone in a reservoir is stimulated (Step 102). The zone can be a zone in the reservoir that can support biodegradation of petroleum to produce biogenerated methane by methanogenesis. To stimulate the microbial conversion, one or more modifiers can be injected into the reservoir. Fluid, including gas and water, can also be injected into the zone to increase the pressure and gas saturation. A critical gas saturation of the zone can be determined. While gas saturation in the zone exceeds the critical gas saturation, gas is produced from the zone to surface (Step 104). The produced gas includes the biogenerated methane and can also include injected gas and/or solution gas from the reservoir. While producing the gas, the production parameters are monitored including the reservoir pressure and an isotopic composition of the produced gas (Step 106). In some implementations, monitoring the isotopic composition of the produced gas includes monitoring stable isotopic compositions of carbon and/or hydrogen for at least one component of the produced gas, e.g., methane or carbon dioxide. Based on the production parameters, at least one of either the stimulation of the microbial conversion is adjusted to control the composition of the produced gas or the gas saturation in the zone is adjusted to control the mobility of the biogenerated methane (Step 108). In some implementations, the bulk composition of the produced gas can be monitored, for example, the amount of methane, carbon dioxide and/or hydrogen included in the produced gas. In other implementations, the monitored production parameters can include the gas production flow rate, which can also be controlled to adjust the stimulation of the microbial conversion and/or the gas saturation. In some implementations, monitoring the production parameters can include monitoring the composition of the produced water and/or oil, which can provide information as to the efficacy of the production operation. Information obtained from monitoring the production parameters can be used to determine what operating parameters can be controlled to adjust the stimulation of the microbial conversion and/or the gas saturation to enhance production of biogenerated methane from the reservoir.

### Identifying a Zone that can Support Biodegradation

A zone that can support biodegradation to produce the desired gas, e.g., methane or hydrogen, can be identified in a subterranean reservoir. One or more samples of fluids (waters, oils and gases) and rocks in the reservoir can be obtained and analyzed. The samples can be obtained by sampling procedures that are known to those skilled in the art. For example, a fluid (liquid or gas) sample can be retrieved from the reservoir through perforations in a well casing or from an open-hole test. The fluids can be sampled either downhole with a wireline formation fluid tester or fluid sampler, or at the surface wellhead from a subsurface test, such as drill stem tests, production tests, or normal production. Both formation water (i.e., water from the reservoir) and petroleum (oil and gas) samples are useful for evaluation of the reservoir environment. Rock samples can be retrieved from drill cores, cuttings, produced sediments and/or outcrop sites or rock data can be secured by interpretation of well logs or other techniques.

An analysis of the reservoir's environment can provide information that can be used to determine suitable microbial growth stimulants or in situ environmental conditions for microbial activity. The analysis can include determining the reservoir's temperature and pressure, which can be obtained in any suitable manner. While many reservoirs contain biodegraded oils, not all reservoirs contain currently active microbial populations. In one implementation, the analysis is to identify a zone in a reservoir that includes relevant active organisms biodegrading reactive petroleum components that can be accelerated to recover economic levels of methane through petroleum biodegradation.

Microbial populations in deep subsurface environments are typically very low and on the order of four to six orders of magnitude less abundant than in near-surface sediments (e.g., 10³ to 10⁴ cells per cubic centimeter in the deep subsurface). Thus to avoid misidentification of contaminant organisms as indigenous, contamination control measures can be adopted for microbiological and microbial ecological analyses. Treatment of all reagents and materials, except amplification primers, with UV and enzymatic treatment with DNase I can be important when nucleic acid based analyses are conducted.

In some implementations, samples for nucleic acid analysis can be frozen immediately or fixed by addition of filtered 50% ethanol. Subsamples can be taken from the centre of whole cores under sterile conditions to avoid contamination from the exterior of the core contaminated during drilling. Samples for cultivation based studies can be stored either chilled or at close to in situ temperatures to reduce the growth of contaminating microorganisms during storage and transport. Samples can be of core material to increase the likelihood of obtaining indigenous organisms free from contaminants. However formation water and/or drill cutting samples can be analyzed for the presence of active microorganisms, if conditions are maintained to inhibit exogenous contaminant organisms while promoting those adapted to in situ conditions. Microorganisms in water samples are preferably concentrated by filtration and/or centrifugation before the analysis is performed. The amount of indigenous microbes will typically be a small fraction of the sample's volume. In a typical oil-bearing reservoir, water may contain less than 0.025 mg of microorganisms per liter. Microorganism concentrations can be amplified to facilitate detection using conventional microbial detection techniques, which are familiar to those skilled in the art. Incubation of samples in microcosms that replicate as much as possible in situ conditions to identify factors that promote or inhibit particular metabolic processes is an approach to identifying candidate petroleum systems for successful microbial stimulation.

To determine the environment in the reservoir, a geochemical analysis can be made of one or more fluids of the reservoir, such as formation water and petroleum, and/or one or more solids of the reservoir, which analyses are familiar to those skilled in the art. The fluid analysis can include measurement of the state values (e.g., temperature and pressure) as well as a geochemical analysis of the formation water, which can include assay for major anions and cations, pH, oxidation potential (Eh), chloride, sulphate, phosphate, nitrate, ammonium ion, salinity, selenium, molybdenum, cobalt, copper, nickel, and other trace metals contents. The geochemical analysis can identify by-products that are known to be produced by indigenous microbial activity. For example, the presence of methane, CO₂, RNA, DNA, enzymes, and/or specific carboxylic acids can be indicative of microbial activity. Methane relatively depleted in the carbon 13 isotope is frequently found in oilfields where natural methanogenesis has occurred. In particular, anaerobic hydrocarbon degradation metabolites, such as alkyl and aryl substituted succinates or reduced naphthoic acids, are markers of systems in which the anaerobic degradation of hydrocarbons is taking place. The identification of such markers can be used in determining the presence of active anaerobic petroleum degrading microbial consortia.

A number of laboratory studies using aliphatic, aromatic, and polycyclic aromatic hydrocarbons as substrates for a variety of sulfate-reducing, denitrifying and methanogenic cultures have identified alkyl and aryl succinates, formed by the addition of fumarate either to a sub-terminal carbon of an alkane or to an alkyl substituent of an aromatic hydrocarbon, as the initial relatively stable metabolite in the degradation process. Succinates have also been reported as metabolites from the biodegradation of both saturated and aromatic hydrocarbons in anoxic zones of petroleum-contaminated aquifers. A study of an anoxic zone in an aquifer contaminated with gasoline identified 2-naphthoic acid and reduced 2-naphthoic acids as evidence of anaerobic degradation. Actively degrading oilfields can contain 2-naphthoic acid and amounts of reduced 2-naphthoic acids, such as 5,6,7,8-tetrahydro-2-naphthoic acid, which are indicators of anaerobic hydrocarbon degradation under the conditions appropriate for methanogenesis. The presence of such compounds can be indicative of anaerobic degradation conditions appropriate for methanogenesis.

Other compounds that are indicative of active methanogenesis under indigenous conditions include archaeols and lipid molecules characteristic of methanogenic archaea. Archaeols characteristic of methanogens can indicate active methanogenesis. Specific phospholipids and microbial DNA characteristic of methanogenic archaea can also be used to identify reservoirs with active methanogenic processes that are capable of acceleration to commercial rates of methane production. In addition methanogens can contain novel co-factors such as F₄₃₀, a nickel porphyrin associated with methyl coenzyme M reductase. A similar, but distinct nickel porphyrin with a higher molecular weight is associated with anaerobic methane oxidizing archaea. Analysis for these components can provide information about the relative prevalence and location of methanogens and methane-oxidizing archaea.

In some implementations, the analyses can be focused on oil-water transition zones in formations. Some specific indicators of active degradation have been shown to be preferentially concentrated in samples near petroleum/water contacts.

Actively degrading petroleum reservoirs can also be identified by several geochemical proxies, for example, elevated carbon dioxide levels in produced gases, isotopically distinct methane enriched in the carbon 12 isotope, acidic metabolite markers as described above and by the detection and measurement of compositional gradients in the oil column. Gradients in oil columns, such as variations in the saturated hydrocarbon contents versus depth in an oil layer, have been detected in several oilfields and can be used to assess the indigenous rates of hydrocarbon metabolism by reservoir microorganisms. The gradients can be produced when organisms destroy petroleum at the base of an oil column and the compositional profile of the oil column changes as a response, thereby producing a vertical and or lateral gradient in composition in such parameters including, but not limited to, saturated hydrocarbon content, n-alkane distribution or content or in the distribution of more resistant compounds such as isoprenoid alkanes or hopanes. The detection of such gradients can be used to identify zones where methanogenesis can be accelerated, as organisms are typically active in reservoirs where gradients are present. The rate of biological activity can be determined from the gradient and thus indicate the extent to which acceleration of natural rates of degradation and methanogenesis are required. This can be used to assess the extent of additive treatments necessary for enhancement of methanogenesis to desired rates.

It is not only organic geochemical signatures that give indications of active processing of the oil naturally by microorganisms. High concentrations of metals such as cobalt, nickel or iron in the oils in the vicinity of the oil/water contacts in a field's column are commonly found in reservoirs where active biodegradation has occurred and may be occurring.

Petroleum analyses can include quantitation of the major hydrocarbon types such as saturated hydrocarbons, aromatic hydrocarbons, resins and asphaltenes and detailed molecular characterization of the specific hydrocarbon fraction such as n-alkanes, isoprenoid alkanes, alkylbenzenes, alkylnaphthalenes and so on. Petroleum geochemical analyses of oil and gas can aid in identifying the abundances and compositions of the different carbon substrates for the microorganisms. While in principal many of the components of crude oils can be used for methanogenesis, the most reactive oils and the oil fields most suitable for methanogenic conversion will generally still include abundant n-alkanes, isoprenoid alkanes and other more reactive components, such as light alkanes and aromatic hydrocarbons. Analysis of petroleum extracted from produced fluids or cuttings or core samples taken through an oil column can allow chemical analyses to define the extent of any compositional gradients that exist in the oil column. Determination of the compositional gradients can be used to determine the current rates of biodegradation of the oil column, the sites of most active biodegradation and thus the extent to which biodegradation rates and methanogenesis rates in particular need to be accelerated.

The rock analysis can include mineralogical, chemical and facies descriptions as well as measurements of reservoir properties such as porosity, permeability, capillary pressure, and wettability.

Analysis of the reservoir geological environment can be carried out using geophysical and geological mapping procedures. The relative volumes and spatial arrangements of oil layers and water layers in the reservoir can control the net rates of biodegradation. Oil zones adjacent to, or surrounded by, reservoir zones saturated with water can be optimal for stimulation. Residual oil zones with high water saturations can be favorable environments for stimulation.

Modify the Reservoir to Stimulate Production of Biogenerated Methane

The zone identified that can support biodegradation to produce a gas, e.g., methane, includes one or more methanogenic microorganisms. The microorganisms are characterized and compared to at least one known microorganism having one or more known physiological and ecological characteristics. If one or more methanotrophic microorganisms (i.e., adverse organisms that promote degradation of the methane) are identified in the zone, then the methanotropic microorganisms are characterized and compared to at least one known microorganism having one or more known physiological and ecological characteristics. The information obtained from the above characterizations and comparisons can be used to determine an ecological environment that will promote in situ microbial degradation of petroleum and promote microbial generation of methane by at least one methanogenic microorganism.

Additionally, in implementations where methanotropic microorganisms are present, the information can be used to determine an ecological environment that demotes in situ microbial degradation of methane by at least one methanotrophic microorganism. The reservoir environment can then be modified based on the determinations to stimulate microbial conversion of petroleum to methane, while minimizing methane destruction by adverse processes.

The following describes one implementation of carrying out the above steps and modifying the reservoir to promote production of biogenerated gas, in an example where the biogenerated gas is methane. Microorganism characterization as used herein means identifying characteristics of a microorganism or consortium of microorganisms using one or more of the following methods: biochemical methods, physiological methods, biogeochemical process measurements, optical methods, or genetic and molecular biological methods. The degree of similarity between these characteristics of sampled microorganisms and microorganism with known properties can be used to identify and infer the physiology, metabolic functions, and ecological traits of the sampled microorganisms by techniques well established in the field of microbial ecology. Non-limiting examples of characterization methods that can be used include:

(a) enrichment culture techniques to obtain microorganism isolates from which biochemical, morphological, physiological, ecological, and genetic traits may be determined and compared against the traits of known microorganisms;

(b) determination of the phospholipid fatty acid composition (PLFA) of the indigenous microorganisms and comparison with PLFA distributions of known microorganisms;

(c) determination of isoprenoid glyceryl ether distributions (archaeols) characteristic of methanogenic archaea and comparison with isoprenoid glyceryl ether distributions of known microorganisms;

(d) compound-specific isotope analysis of microbial lipids to identify organisms utilizing methane;

(e) characterization of specific nickel porphyrins to distinguish methanogenic and methane-oxidizing archaea;

(f) genetic characterization methods, of which two non-limiting examples (among many) include the following:
(1) Sequences of genetic fragments from sampled microorganisms including but not restricted to 16 S rRNA genes (bacterial, archaeal) encoding the alpha subunit of methylcoenzyme M reductase (mcrA) from methanogenic and methane-oxidizing archaea and genes encoding the alpha-subunit of benzylsuccinate synthase (bssA) and homologues, involved in the initial activation of hydrocarbons by anaerobic hydrocarbon degrading bacteria. These can be compared against nucleic acid sequences from known microorganisms, for example, using the Ribosomal Database Project, Michigan State University, East Lansing or the Genbank database at the National Center for Biotechnology Information located in the National Library of Medicine (Building 38A Room 8N805), Bethesda, Md. 20894, U.S.A., to establish the phylogenetic identity with nearest known relatives using established techniques. In particular, quantitative analysis of these target genes characteristic of particular organisms or processes that can be controlled for enhancing recovery of methane (e.g., using real-time PCR) is of use, as is the design of specific primers that can be used to distinguish and quantify the variants of mcrA involved in methanogenesis and methane-oxidation respectively and for quantification of potential primary hydrocarbon-degrading syntrophs; and
(2) Oligonucleotides designed to hybridize to the 16 S rRNA genes of specific microorganisms and target genes indicative of key processes (hydrocarbon activation, methane generation, methane oxidation) can be used in polymerase chain reaction-based methods. Although potentially applicable, the use of such oligonucleotide probes labeled with radioactive phosphorus, biotin, fluorescent dyes, enzymes and other suitable tags may lack the sensitivity required for analysis of subsurface samples, unless linked to amplification techniques such as the polymerase chain reaction or culture-based enrichment or analysis of microcosms.

From knowledge of the indigenous microorganisms and their nutritional requirements, the chemical composition of the reservoir's oil, water and matrix rock, and the physical characteristics of the reservoir (pressure, temperature, porosity, saturation, etc.), the overall ecological environment needed to promote and retard the activity of appropriate members of the microbial consortium present in the reservoir can be determined. This information can be then used to modify environmental conditions in the reservoir to promote microbial conversion of petroleum to methane and to prevent microbial degradation of the methane thereby produced.

Altering the activity of microorganisms in the subsurface can depend on at least one of the following factors: (1) adding and/or subtracting and/or maintaining components required for microbial growth and/or activity as determined by the laboratory and/or in situ pilot studies; and (2) controlling and/or maintaining the subsurface environment (for example, chemistry, temperature, salinity, and pressure).

Microbial Ecology

To accelerate methane production it can be desirable to accelerate the activity of syntrophs and methanogens, while reducing methanotroph activity. To convert petroleum to methane, the reservoir's indigenous microbial consortium may include petroleum-degrading microorganisms having similar genetic characteristics to one or more of microorganisms listed below. If hydrocarbon degrading iron-reducing, nitrate-reducing (including, but not exclusively, denitrifiers), sulphate-reducing bacteria and/or archaea are present, specific steps can be taken to inhibit their activity, otherwise hydrocarbons may be degraded to carbon dioxide and water without the formation of methane. Furthermore, aerobic hydrocarbon degrading organisms identified are unlikely to be indigenous to the reservoir. These too can be detrimental to the process of petroleum hydrocarbon conversion to methane. Such organisms will most likely be inactive, unless substantial quantities of oxygen are provided.

Potential syntrophic organisms that will convert complex organic carbon in petroleum into substrates that can be converted to methane by methanogens include organisms related to the following: Syntrophobacter spp., Syntrophus spp., Syntrophomonas spp., Thermoanaerobacter and relatives, Thermotoga, Thermoanaerobacterium, Fervidobacterium, Thermosipho, Haloanaerobium, Acetoanaerobium, Anaerobaculum, Geotoga, Petrotoga, Thermococcus, Pyrococcus Clostridium and relatives, and the reservoir may also include methanogenic archaea of one or more of the orders Methanobacteriales, Methanomicrobiales, Methanosarcinales and relatives, Methanopyrales, and Methanococcales to convert syntrophic organism degradation products to methane.

Organisms that may result in lower methane yields may also be present in the reservoir and can be identified. These include, for example, anaerobic methane oxidizing archaea (methanotrophs), referred to as ANME-1 and ANME-2, which are related to but distinct from the Methanosarcinales. In addition to these two major groups of methane-oxidizing archaea, other groups may be present. If present, the activity of such organisms can be controlled to prevent reduction in methane production.

Understanding the subsurface ecology allows one skilled in the art to deduce likely modifiers that can stimulate subsurface activity. Modifiers can include (in an appropriate form for distribution throughout the reservoir) but are not limited to:
Major nutrients containing nitrogen and phosphorus that do not accelerate competing processes, such as nitrate or sulphate reduction. Non-limiting examples include: Na₂HPO₄, K₂HPO₄, Na₄Cl added via water injection or ammonia gas (NH₃), or volatile phosphorus (PH₃, CH₃—PH₂) compounds added, which can be quickly dispersed through the gas caps, facilitating nutrient supply quickly over large areas of the fields. Phosphates may precipitate chemically in reservoirs and therefore less reactive forms of phosphorus, such as polyphosphate and phosphorus pentoxide may be appropriate additives; NaNO₃, KNO₃, NH₄NO₃ can accelerate the syntrophic components of methanogenic consortia, however, methanogens exclusively use ammonium ion as a nitrogen source and addition of nitrate can stimulate nitrate reducing bacteria, which can repress methanogenesis by more effective competition for electron donors. Therefore the correct form of nitrogen and phosphorus can be added in order that processes that would inhibit methanogenesis are not fortuitously stimulated.
Vitamins, including, for example, cyanocobalamine (vitamin B₁₂), folic acid, ascorbic acid, and riboflavin.
Trace elements including, for example, B, Zn, Cu, Co, Mg, Mn, Fe, Mo, W, Ni, and Se.
Buffers for environmental controls.
Waters of different salinities and pH values or containing complexing agents such as organic acids such as oxalate, EDTA or other multidentate ligand organic compounds, including hydroxylated acids, to facilitate mineral dissolution and release of natural nutrients including, but not limited to, potassium, ammonium or phosphate ion from dissolution of feldspars, clays or other silicates and carbonates.

Modifiers can be used to accelerate methane production. For example, if cobalt or nickel is known to stimulate growth of the closest-matching methanogenic microorganisms, and if cobalt or nickel is present in the reservoir in only limited concentrations in a labile accessible form, then addition of these limiting components in an accessible soluble form to the reservoir can also stimulate the uncharacterized methanogens.

Suitable stimulants can be tested and optimized using indigenous microorganisms in laboratory microcosms, cultures or in situ pilot sites to determine their effectiveness at promoting rapid petroleum-degradation and methanogenesis. However, stimulants chosen should not increase the rate of activity of any methanotrophic or nitrate, iron or sulphate reducing microorganisms that will suppress methanogenesis by competition for common electron donors. If such organisms are stimulated, preferably their activity is independently blocked.

Indigenous microbial consortia can be grown in nutrients using a range of nutrient media, with varying pH, salinity, trace metals, to find the conditions that support high rates of petroleum degradation linked to methanogenesis and low rates of methane degradation. These microcosm and culture studies can involve several cycles of stimulant addition and stimulant combinations as well as varied environmental conditions (e.g. salinity, temperature, pH see below). Because the indigenous microorganisms found in a given reservoir and the chemistry of the reservoir fluids and reservoir rocks will typically be unique to that reservoir, the conditions for promoting growth of indigenous microorganisms can vary from one petroleum accumulation to another, and can vary from one location in the petroleum accumulation to another. Conditions favorable for microorganism growth in part of the petroleum accumulation may not be optimal for another part of the petroleum accumulation. In addition it may be necessary to inhibit methane-oxidizing archaea that are present in locations that are removed from the site of methane generation, to minimize loss of methane during extraction from the reservoir.

Hydrocarbon degradation in deep subsurface petroleum reservoir environments is often phosphorus, potassium or nitrogen limited. Studies have shown a close relationship between the geomicrobiology of petroleum-contaminated aquifers, mineral alteration and groundwater chemistry. Biological activity perturbs general groundwater chemistry and therefore mineral-water equilibria, and at the microscale, attached organisms locally perturb mineral-water equilibria, releasing limiting nutrients. In an oil-contaminated aquifer, it has been shown that feldspars weather exclusively near attached microorganisms in the anoxic region of the contaminant plume, and that indigenous bacteria colonized feldspars that contain potassium or trace phosphorus. Most phosphorus in many petroleum reservoirs and reservoir encasing sediments is in feldspars and it has been suggested that natural feldspar dissolution in some oil reservoirs is related to biodegradation of the associated oils. Phosphorus contents of oils are low (approximately 1 ppm or much less) whereas phosphorus contents of sandstone reservoirs or reservoir encasing shales are much higher (up to 1000 ppm or more of low solubility oxide equivalents). The phosphorus is thus generally present in mineral phases of low water solubility. Supply of limiting nutrients from mineral dissolution in reservoirs or reservoir encasing shales in many instances may be the rate-limiting step in subsurface petroleum biodegradation. Addition of phosphorus as soluble forms of phosphates in injected waters or alteration of reservoir water chemistry by change of pH, salinity or addition of complexing agents including organic acids or multidentate organic chelating agents can be used to release available phosphorus or potassium from minerals to accelerate petroleum biodegradation. Ammonium phosphate or potassium ammonium phosphate can add both nitrogen and phosphate and also potassium.

The concentration of ammonium ion (NH₄⁺) in the formation waters can also affect the rate of methanogenesis. Naturally, in petroleum reservoirs, mean concentrations of ammonium ion range from a few ppm up to a up around 500 ppm, but are typically around a few tens of ppm. By contrast, in near surface anoxic environments (e.g., landfills), concentrations of ammonium ion range up to over 1000 ppm. Nitrogen supplied in the form of ammonium ion can accelerate methanogenesis, whereas if supplied as nitrate, competitive nitrate dissimilatory reduction can eliminate or reduce methane production.

In sandstone reservoirs (i.e., reservoirs in which petroleum is trapped in the pore systems of sandstones), the concentration of nutrients, such as phosphorous, can be rate limiting on overall oil biodegradation rate and methanogenesis. The concentration of phosphorous can be increased by the addition of exogenous phosphorous, or by release of phosphorous from the reservoir matrix by modifying the characteristics of the reservoir waters, such that the phosphorus containing minerals in the reservoir, such as clays or feldspars, dissolve releasing their phosphorus. For example, injection of fresh, low salinity waters or acidic waters can aid in feldspar dissolution releasing nutrients.

The addition of organic acids such as oxalate, EDTA, citrate or other multi-ligand chelating agents including hydroxylated acids and other multi functional chelators can also facilitate mineral dissolution and release of natural phosphorus and other nutrients from reservoir minerals. These treatments can stimulate all the microorganisms present, not only those required for conversion of petroleum to methane. To prevent the activity of organisms that will out compete methanogens for electron donors, certain amendments can be required to suppress their activity. These amendments can include (but are not limited to) sodium molybdate (or other hexavalent cations) to inhibit sulphate-reducing bacteria and sodium chlorate to inhibit nitrate reducing bacteria. Methane-oxidizing archaea are unlikely to be active at the site of methanogenesis, but if present in other regions of the reservoir, can be inhibited. The fact that these groups of archaea are likely to be spatially separated can be important, since the known inhibitors of anaerobic methane oxidation (e.g., bromoethane sulfonic acid) also inhibit methanogens. In addition methane-oxidizing archaea often exist in close association with sulphate-reducing bacteria that consume the products of anaerobic methane oxidation driving methane oxidation to completion. This permits anaerobic methane oxidation to be inhibited with inhibitors of sulphate reduction such as sodium molybdate.

Reservoir Conditions

Environmental conditions in petroleum bearing, subterranean reservoirs may not be conducive to thriving populations of the appropriate indigenous microorganisms. The appropriate microorganisms may need to be stimulated to be more active. This stimulation can be carried out by modifying one or more parameters of the reservoir environment. For example, high-salinity environments may greatly slow the rates of petroleum degradation and methanogenesis. Introduction of low salinity waters may stimulate the degradation and methanogenesis activity. The environment can also be altered to slow the rate of methane degradation. Preferably, the changes required to increase the rates of petroleum degradation and methanogenesis will simultaneously decrease the rate of methane degradation.

In general, for implementations promoting the production of biogenerated methane, the methods and systems described can be practiced in any petroleum-bearing reservoir that is suitable for microbial life, or that can be modified to be suitable for microbial life. In general, the reservoir fluids will have a temperature less than about 130°Celsius (C), a pressure less than about 80,000 psig (55160 kPa), a subsurface pH between about 3 and 10, and a salt concentration less than about 300,000 parts per million (with preferred values less than 150,000 parts per million). Typically however, normally pressured reservoirs cooler than 80° C., or which can be cooled to below 80° C., with salinities less than 50000 parts per million are the optimal reservoirs for treatment. Indigenous syntrophic and methanogenic organisms are not likely to be active in reservoirs hotter than 80° C., or where geochemical and geological data indicate the reservoir has ever been heated to more than 80° C. In these circumstances injection of exogenous methanogenic consortia may be necessary.

Reservoir environmental parameters of principal concern for providing optimal petroleum degradation and methanogenesis conditions include, but are not limited to, temperature, salinity, pH, alkalinity, organic acid concentration, nutrients, vitamins, trace elements, availability of terminal electron acceptors (high levels will suppress methane generation), and toxic substances (to suppress the activity of competing microorganisms). One or more of these environmental parameters may require adjustment or maintenance within specific ranges to initiate or sustain commercial rates of methane generation.

The environmental conditions for promoting growth of a microbial consortium in a reservoir can involve many factors including, without limitation, the following, either alone or in combination: (1) changes in the reservoir temperature, pH, Eh, mineralogy, and salinity and the concentrations of CO₂, O₂, and H₂ in the reservoir; and (2) creation, movement and/or maintenance of water-oil interfaces between different petroleum-degradation microbial populations, and/or microbial methanogenesis zones.

Modifying the Reservoir Environment

A stimulant and/or inhibitor added to the reservoir and/or a change of one or more environmental factors (either alone or in combination) are referred to herein as microbial growth “modifiers”. A particular modifier, or combination of modifiers, suitable for a particular application depends on the microbial consortium to be modified and the reservoir environmental conditions. Since indigenous microorganisms are typically in a nutrient deprived state, one stimulation strategy involves the addition of a nutrient. However, since stimulating methane production is also likely to stimulate adverse processes including methane degradation, the modifier package can include an inhibitor for adverse activity, as described above. Once a modifier package is determined, the reservoir environment can be altered on a continuing basis, or discontinued after a suitable period of time, to permit change in the populations of the microorganisms, depending on assessment of environmental analyses of the producing reservoir. As mentioned above, in fields where there is no activity of the indigenous microorganisms, the addition of exogenous microorganisms may be necessary. These may also be referred to as “modifiers”.

Injection Process

For growth or activity modifiers that involve injecting a material into the reservoir, the material can be added to a fluid flood such as an aqueous solution, gas (such as CO₂), solvent or polymer that is injected into the reservoir by any procedure found convenient. In some implementations, the modifiers can be introduced into the reservoir by a waterflood program. To simplify the following discussion, the above-identified injection carrier will be referred to as water, although other carriers can be used. The amount of water introduced into the reservoir and the amounts of microbial modifiers contained in the water can depend upon the results desired.

Multiple modifiers can be injected into the reservoir together or in separate injection steps. For example, a slug or bank of water carrying one modifier can be followed by a second slug or bank of water carrying a second modifier. Another example may include alternately injecting one water bank followed by a gas injection step. In some implementations, modifiers operating as stimulants may be injected at one location to enhance methanogenesis, and modifiers operating as inhibitors may be injected at a different location, to prevent or minimize detrimental processes, such as methane oxidation. Injection of gas below a degrading oil column may facilitate circulation of water and nutrients to the microorganisms present, and may also allow for injection of volatile microbially accessible nutrients, which can disperse rapidly in a gas phase in the reservoir environment.

In some implementations, layered reservoir bioreactors can be used for methane production and to facilitate methane removal. In such a reservoir bioreactor, the biodegrading oil column and/or residual oil zones are vertically segmented and the environment can be controlled, for example, in the following manner: (a) a lower zone of degradation of oil or injected reactive organic substrates can be environmentally modified to produce abundant free gas (e.g., methane and/or carbon dioxide); (b) an upper zone of degradation of oil or injected reactive organic substrates is environmentally modified to produce abundant free methane; (c) free gas from the lower layer buoyantly moves up through the layered bioreactor and any free methane or methane in aqueous or oil solution partitions into the moving gas phase and is carried to a gas-rich zone for production.

Gas flushing or sparging of degrading oil columns by injecting gas from a well or by producing gas in a biodegrading reservoir layer below the zone to be flushed can also be employed. A gas phase (methane, carbon dioxide, and air) can be injected below the degrading oil column. With methane and carbon dioxide, simple partitioning occurs and removes methane as a free gas phase. With air, aerobic degradation of organic matter at the base of the column removes oxygen and facilitates pressure increases and production of large volumes of gas (carbon dioxide) to move up into an anaerobic zone where methane production is occurring. Gas sparging or flushing of degrading oil or residual oil zones can facilitate introduction of nutrients, either as entrained water soluble nutrients or via volatile gas transported nutrients. This can be a fast way of getting nitrogen, phosphorous and other nutrients to the methane production zones. Gas sparged or flushed reservoirs, or reservoirs operating without gas sparging, can have injector wells below the initial oil-water contact (OWC) to inject nutrients, inhibitors and metabolic modifiers into waters that can migrate up into the degrading oil zones as production proceeds. Also gas sparged zones where the gas saturation certainly exceeds the critical gas saturation can be used to accelerate gas transport from lower regions of the reservoir to the top of the reservoir where the gas can accumulate as a gas cap or in a gas rich zone.

In some implementations, acceleration of methanogenesis, provision of nutrients, injection of organic matter-degrading microorganisms and production of gases (methane and carbon dioxide) can be facilitated by injection of reactive liquid organic matter into or below biodegrading oil legs. Organic matter may be from sewage, waste waters, biomass (e.g., liquid waste) and industrial chemical wastes and farm wastes among others. Such materials can be injected as part of a normal reservoir pressure maintenance program into actively degrading petroleum columns, or into sterile petroleum reservoirs needing infection with organic matter degrading organisms. To accelerate degradation of reactive organic matter such as sewage for gas production (in the form of carbon dioxide) and pressure production, NaNO₃, KNO₃, NH₄NO₃, can be suitable additives, however addition of nitrates will potentially inhibit methanogenesis. Accordingly, there can be controls and balances so as to use nitrates to generate carbon dioxide, but so as not to inhibit the methanogenesis.

Creation/Maintenance of Biodegradation Interfaces

Microorganisms in subterranean reservoirs tend to be most active at environmental boundaries, such as between fermentation zones and methanogenesis zones. Therefore, microorganism activity in a reservoir may be increased by increasing the number of such boundaries, which serve as environmental interfaces. One method for increasing the number of environmental interfaces is to modify the water flood injection rates. A second method is to alternate or vary the injection modifiers into the reservoir to create, in effect, moving environmental fronts. A third method involves forming small-scale environmental interfaces by forming petroleum-water emulsions in the reservoir, or by changing the clay chemistry. A fourth method relies on knowledge of field geometry. The optimal fields for processing for methanogenesis are fields where already existing natural interfaces between water and oil are large. These include any fields with residual oil columns, where the pore space is only partially oil-saturated, or contains much more water than the critical water saturation point, produced either naturally over geological time or via primary or enhanced recovery procedures. Fields with artificially introduced high permeability highly water saturated zones, such as wormholes, produced during oil recovery by Cold Heavy Oil production with Sand (CHOPS), can be particularly optimal.

Oil fields that have large residual oil columns below the producible oil legs can provide an optimal environment for producing biogenerated methane A common process during field filling is movement of oil legs through field tilting, oil leakage through seals and during the biodegradation process oil is naturally consumed, and oil legs move upwards leaving a residual oil zone with large water/oil interfacial areas. Some of the best fields for biological recovery of oil as methane, such as the Troll field or Frigg field in the North Sea, often have thick natural residual oil zones with high water saturations, which can be preferred for processing methane through microbial activity.

Injection wells can be located below the oil water contacts or below residual oil zones that migrate upwards during normal oil production or consumption of oil during biodegradation and allow the oil zone to move upwards, to facilitate movement of water through any residual oil remaining. This can allow for modifiers and organisms to be dispersed upwards into the remaining oils, facilitating increased degradation rates and methane production.

As an example of changing environmental conditions, oil formation waters often contain low concentrations of indigenous phosphate ion, which can be a rate controlling nutrient in some biodegrading reservoirs. Injecting water of very low salinity, or with a pH different from the reservoir pH or waters, containing organic acids, such as oxalate or citrate or other complexing agents, can aid in dissolution and release from minerals, such as feldspars or clays, of nutrients such as phosphorous, nitrogen, potassium, cobalt or nickel. Alternatively phosphorus can be added as phosphate, polyphosphate or phosphorus pentoxide, nitrogen as ammonium ion or urea and potassium, cobalt or nickel as water soluble salts.

During the injection process for stimulating microbial transformation of petroleum to methane and inhibiting microbial degradation of methane, both the reservoir conditions and the microbial dynamics (ecology) can be monitored. The monitoring can be performed in any suitable manner. Fluid (e.g., oil, gas, and water) samples can be obtained from the reservoir through one or more wells in communication with the reservoir. The samples can be analyzed to determine the concentration and type of microorganisms in the fluid, as well as the concentration of modifiers and microbial products in the fluid. Other geochemical analyses also can be performed to assess the effectiveness of the stimulants on the reservoir environment and to confirm the chemical compatibility of the desired component to be injected and the subsurface fluids and solids. If based on this geochemical monitoring, the modifier effect in the reservoir is outside the desired range, the concentration of modifier being injected can be adjusted to bring the modifier concentrations to within an acceptable range.

### Injecting Fluid into the Zone

Although solution gas (i.e., gas dissolved in a liquid oleic phase) is often produced along with oil when producing oil, the goal of oil production is to maximize the amount of oil produced. By contrast, the methods and systems described herein are directed to enhancing the production of a biogenerated gas from a reservoir. As discussed above, the reservoir can include oil that is not recoverable by conventional oil production techniques. However, when focusing on enhanced gas production, the goal is not to produce the oil from a production well, but rather to produce the biogenerated gas, preferably in a gas phase. Some liquid phase, including oil and water, may be produced along with the gas, and the liquid phase may include gas dissolved therein, which gas may include the biogenerated gas.

Increasing the pressure in the reservoir prior to production of biogenerated gas from the reservoir can promote partitioning of the biogenerated gas into solution (i.e., dissolved in reservoir fluid). If the generation of methane is sufficiently large, the storage capacity of the fluid in the reservoir (i.e., the solubility of methane in the oil and/or water phase times the volume of available oil and/or water) can be exceeded and yield free gas within the reservoir. Once the free gas saturation exceeds the critical gas saturation of the reservoir, the gas phase is mobile.

In addition to injecting modifiers into the reservoir to promote microbial generation of methane, fluid including water and gas can be injected to control the reservoir pressure or to promote the gas saturation to reach the critical gas saturation, or a threshold percentage of the critical gas saturation, as shall be explained further below. Critical gas saturation is defined as the gas saturation where gas bubbles become sufficiently connected and the gas phase in this state of connectedness becomes mobile in the reservoir under production conditions. If the gas saturation is lower than the critical gas saturation, the gas phase is immobile (or less mobile). The fluid can be injected either directly into the zone identified as able to support biodegradation of petroleum to form methane, or can be injected into a nearby region, since the fluid can migrate into the biodegradation zone.

Water is an example of the fluid that can be injected, although other fluids are possible. The water can be formation water, i.e., water produced from the reservoir, or water from a different source, e.g., aquifer, different reservoir, or zone at different elevation.

The gas can be injected as free gas and/or can be injected dissolved in the injection water. In some implementations, the injection water is brought to reservoir pressure at surface and is saturated with the gas. The gas-saturated injection water is then injected into the reservoir. Because the injection water was saturated with the gas at reservoir pressure, once injected, the water does not absorb additional gas in situ. Examples of surface facilities for saturating the water with gas include bubble columns and pipelines where liquid and gas mix by using static mixers. In some implementations, the injection water has a similar salinity to the existing formation water, which can either be artificially induced or natural if formation water is being recirculated and re-injected. The temperature of the injection water can be similar to the reservoir temperature.

The gas can be any gas that produces a free gas phase at reservoir conditions. In one implementation the gas is carbon dioxide. In another implementation, the gas is nitrogen or methane or mixtures of such gases. Preferably, the injected gas exists in a free gas state in the reservoir after the reservoir de-pressurizes, providing additional gas accelerating the gas saturation towards the critical gas saturation.

In some implementations, the fluid injected to increase the reservoir pressure and accelerate reaching the critical gas saturation can be injected at the same time as the modifiers are injected to modify the reservoir environment to stimulate microbial conversion of petroleum to methane. In one example, gas-saturated water can be injected that includes the modifiers. In another example, water including the modifiers can be injected with free gas. In yet another example, gas-saturated water that includes the modifiers can be injected with free gas. In other implementations, water including the modifiers is injected first, followed by an injection of gas-saturated water or water and free gas. In other implementations, water including the modifiers is injected at the same time as the water and gas, however from different wells and into similar or different regions in the reservoir. In one implementation, de-oxygenated gas-saturated water at reservoir conditions is co-injected with the modifiers, which can promote growth and methanogenesis as well as accelerate the gas saturation toward the critical gas saturation.

The length of the injection phase can depend on the amount of modifiers and fluid that must be injected into the reservoir to stimulate microbial conversion of petroleum to methane and to raise the reservoir pressure and gas saturation to desired levels. The injection phase refers to the phase during which the modifiers are introduced into the zone, and the phase where the water and gas are injected into the zone, whether these events occur simultaneously or concurrently. In one illustrative example, the injection phase endures for approximately 1 year.

In one implementation, fluid including water and gas is injected into the reservoir until the critical gas saturation in the zone is reached or exceeded. However, once a well is put into production, pressure in the reservoir starts to decline, with the largest amount of decline in the vicinity of the production well. Once the pressure declines, gas, e.g., methane, stored in the liquid phase (i.e., oil) exsolves, forms gas bubbles and merges with existing free gas thereby increasing the gas saturation. If the increase in the free gas as a result of one or more wells going into production can be determined, then the impact on the zone's gas saturation can also be determined. That is, if the known effect on the gas saturation can be determined, then the one or more wells can go into production before the zone reaches the critical gas saturation, so long as the increase in gas saturation as a result of the one or more wells going into production will result in the zone reaching the critical gas saturation once going into production. A “threshold gas saturation” of the zone can be determined at which one or more wells can go into production, such that the gas saturation once in production will reach or exceed the critical gas saturation. For example, depending on the pressure changes with time, the range of the threshold gas saturation can be between approximately 85% and 95% of the critical gas saturation.

### Determining Critical Gas Saturation of the Zone

As discussed above, once the gas saturation in the zone reaches or exceeds the critical gas saturation, the gas phase is mobile. Any convenient technique can be used to determine the critical gas saturation of the zone. The critical gas saturation can be determined by calculation, measurement, estimation, inference or by another convenient manner. Some example techniques are described below.

In one implementation, a material balance method is used to determine the critical gas saturation. The material balance method accounts for the injection and production of oil, gas, and water phases from the reservoir, taking the initial volumes of each phase into account. Since the pore space of the reservoir changes little with time, the sum of the volumes of the three phases at any instant is generally constant throughout time. The material balance (or rather “volume” balance) equation can be written as follows:

PVᵢ=PV_{gi}+PVₒᵢ+PV_{wi}=PV_{gt}+PVₒₜ+=PVₜ

where:
PVᵢ and PVₜ are the pore volumes at the initial state and at time t, respectively
PV_{gi}, PVₒᵢ and PV_{wi} are the pore volumes associated with the gas, oil, and water phases at the initial state; and
PV_{gt}, PVₒₜ, and PV_{wt} are the pore volumes associated with the gas, oil, and water phases at time t.

The material balance method uses pressure and production data for the reservoir, as well as PVT data describing the reservoir fluid behavior as the pressure and temperature changes. If such data are not known for the particular reservoir of interest, the data can be estimated using available data from a closely proximate reservoir having similar properties. The material balance method is a simple approach that treats the reservoir as a single closed volume with constant properties, e.g. pressure, throughout the volume. When each of the oil, gas and water volumes are taken into account in the reservoir, the above formula can be converted to:

$${S}_{g} = [ 1 - ( 1 - \frac{{N}_{p}}{N} ) \frac{{B}_{o}}{{B}_{oi}} + \frac{( {W}_{p} - {W}_{i} ) {B}_{w}}{N \cdot {B}_{oi}} + \frac{( {G}_{p} - {G}_{i} ) {B}_{g}}{N \cdot {B}_{oi}} ] ( 1 - {S}_{wc} ) .$$

where:

S_{g} is the gas saturation at time t;

N is the initial oil volume in place;

Nₚ is cumulative volumetric production of oil up to time t;

Wₚ is cumulative volumetric production of water up to time t;

Gₚ is cumulative volumetric production of gas up to time t;

Wᵢ is cumulative volumetric injection of water up to time t;

Gᵢ is cumulative volumetric injection of gas up to time t;

Bₒ is the oil formation volume factor at time t;

Bₒᵢ is the initial oil formation volume factor;

B_{w} is the water formation volume factor at time t;

B_{g} is the gas formation volume factor at time t;

S_{wc} is the initial (connate) water saturation in the reservoir.

The value for N can be estimated from historical data known about the reservoir. the values for Nₚ, Wₚ and Gₚ can be estimated from production data. The values for W and Gᵢ can be estimated from operation data. The values for Bₒ, Bₒᵢ, B_{w} and B_{g}, i.e., the formation volume factors, are all functions of pressure.

The critical gas saturation can be estimated as the gas saturation at which free gas production occurs, i.e., gas production volumes above the amount that would be expected from solution gas. Before the critical gas saturation is reached, the gas production is simply the amount of solution gas dissolved in the produced oil. After the critical gas saturation is reached, produced gas includes both the solution gas dissolved in the produced oil and also free gas that is now flowing in the reservoir towards the production well. The above formula can be used to estimate the gas saturation in the reservoir after the pressure in the reservoir has dropped below the bubble point pressure. The formation volume factors are all functions of pressure, and therefore the gas saturation is given as a function of pressure. At this pressure, gas is starting to come out of solution and is forming gas bubbles in the reservoir, but is not flowing since the gas saturation is less than the critical gas saturation (i.e., the bubbles are not connected). As the pressure drops further (e.g., because of production), the gas saturation rises and at some point gas production exceeds the amount expected from solution gas dissolved in the oil. The gas saturation where this occurs provides an estimate of the critical gas saturation.

In another method, pressure, production and PVT data can be used with a reservoir simulation model of the reservoir to determine the critical gas saturation. The reservoir simulation model can treat the reservoir as subdivided gridblocks each with its own property set. It allows a more refined and heterogeneous description of the reservoir than that of the material balance method. The critical gas saturation of the gas-liquid or gas-oil relative permeability curves can be adjusted until the reservoir simulation yields substantially the same oil, water, and gas production rates as that from the field operation (i.e., the pressure and production rates or volumes). Other parameters, e.g. permeability, porosity, etc. of the reservoir model also can be adjusted to obtain this match.

In another implementation, particularly if production data are not available, the critical gas saturation can be estimated by laboratory tests. A sample of the reservoir containing fluids restored to reservoir conditions can be de-pressurized by producing liquids in the laboratory until gas flow production begins. Based on the amounts of oil and water and eventually gas produced, as well as the pore volume of the sample, the critical gas saturation can be determined.

### Determining when to Commence Methane Production

Production of methane from the zone to surface can commence once the zone's gas saturation reaches or exceeds the critical gas saturation, or the threshold gas saturation, as described above. To determine when the gas saturation satisfies this condition, the zone's gas saturation can be monitored during the injection phase or during an optional soak period. The gas saturation can be measured, calculated, estimated, inferred or otherwise determined. In some implementations, the zone is allowed to soak for a period between the injection phase and the production phase to allow time for the modifiers to have an effect on the zone and for the volume of biogenerated methane to increase.

In some implementations, the zone's gas saturation can be determined based on pressure measurements taken from the reservoir. For example, the free gas saturation in the reservoir can be determined by the following formula:

$${S}_{g} = [ 1 - ( 1 - \frac{{N}_{p}}{N} ) \frac{{B}_{o}}{{B}_{oi}} + \frac{( {W}_{p} - {W}_{i} ) {B}_{w}}{N \cdot {B}_{oi}} + \frac{( {G}_{p} - {G}_{i} ) {B}_{g}}{N \cdot {B}_{oi}} ] ( 1 - {S}_{wc} ) .$$

where:

S_{g} is the estimated gas saturation at time t;

N is the initial oil volume in place;

Nₚ is cumulative volumetric production of oil up to time t;

Wₚ is cumulative volumetric production of water up to time t;

Gₚ is cumulative volumetric production of gas up to time t;

Wᵢ is cumulative volumetric injection of water up to time t;

Gᵢ is cumulative volumetric injection of gas up to time t;

Bₒ is the oil formation volume factor at time t;

Bₒᵢ is the initial oil formation volume factor;

B_{w} is the water formation volume factor at time t;

B_{g} is the gas formation volume factor at time t;

S_{wc} is the initial (connate) water saturation in the reservoir.

The value for N can be estimated from historical data known about the reservoir. the values for Nₚ, Wₚ and Gₚ can be estimated from production data. The values for Wᵢ and Gᵢ can be estimated from operation data. The values for Bₒ, Bₒᵢ, B_{w} and B_{g}, i.e., the formation volume factors, are all functions of pressure. That is, the values for the formation volume factors can be derived from the reservoir pressure. Accordingly, using the known production and operation data, the gas saturation can be estimated as a function of pressure.

In other implementations, the zone's gas saturation can be determined based on continuous logging of the reservoir. If the wells are regularly or continuously logged, then in intervals where the neutron porosity and density porosity logs cross over, there is an indication that a gas-saturated zone exists in that interval. Many log combinations can produce gas saturation estimates. For example if the porosity and subsurface densities of mineral, water and oil phases are known, or can be estimated, and the water saturation is known (e.g. from a resistivity log), then the density log can be used to determine the gas saturation.

In other implementations, the zone's gas saturation can be determined based on an analysis of a reservoir sample extracted from a well. The volume of gas in the sample can be determined as a function of pressure and the gas saturation thereby determined The sample should be preserved at reservoir conditions and brought to surface. Then, at the reservoir pressure and temperature, each phase can be displaced from the sample container. Based on the volumes of each phase, the gas phase saturation can be estimated as a function of pressure.

The following examples are provided for illustrative purposes. The examples demonstrate the impact of injecting fluid including modifiers and gas into the reservoir prior to or in conjunction with gas production from the reservoir. The examples are based on reservoir simulation modeling.

### Example 0

### No Gas Injection

In this example embodiment, two wells are used as shown in FIG. 2. The properties of carbon dioxide, hydrogen, methane, and water including molecular weight, critical pressure and temperature, heat capacity correlations, and viscosity correlations have been obtained from standard, publicly available databases. Standard diffusion and dispersion coefficients have been used. In the heavy oil reservoir matrix, a standard set of oil-water and gas-liquid relative permeability curves, typical of that of heavy oil/bitumen systems was used. The commercially available thermal reservoir simulation software package STARS™ by Computer Modeling Group Ltd. was used for the reservoir simulations. In this example, methane-producing microbial consortia are present within the rock system in the reservoir.

In this example, a water and nutrient mixture is injected into the reservoir through a horizontal well 202. The reservoir, wells, fluids, and microbial processes in the reservoir are modelled by using a reactive reservoir simulator. The microbial processes are represented by a set of chemical reactions with an accompanying set of reaction parameters and constants. The simulator solves for fluid velocities, pressure, fluid saturations, and component concentrations.

The lengths of the injection and production horizontal wells are 1000 m each. The horizontal permeability and porosity of the reservoir is 5 mD and 0.33, respectively. In this example, the nutrient and water mix is injected at a constant pressure of 4000 kPa into the reservoir. The water plus nutrient mixture flow rate is on average equal to about 7 m³/day is injected for 365 days. The initial pressure of the reservoir prior to injection is 1590 kPa. The initial oil and water saturations are equal to 0.65 and 0.35, respectively. Initially, there is no free gas in the reservoir. The initial temperature and pressure of the reservoir is 10 degrees Celsius. At initial conditions, the reservoir consists of an oil-water system, so there is no free gas phase present.

The bottom well is a horizontal injection well 202 that injects nutrient plus water into the reservoir. The top well is a horizontal producer well 204 that produces fluids, including biogenerated gas, from the reservoir. The two wells operate simultaneously, in other words, injection of the nutrient mix occurs at the same time as production of reservoir fluids. In the reservoir, while the gas saturation is lower that the critical gas saturation, the gas does not flow readily to the production well. During methanogenesis, the biogenerated gas adds to the gas saturation and when the total gas saturation exceeds the critical gas saturation, then the gas becomes mobile and moves in the reservoir.

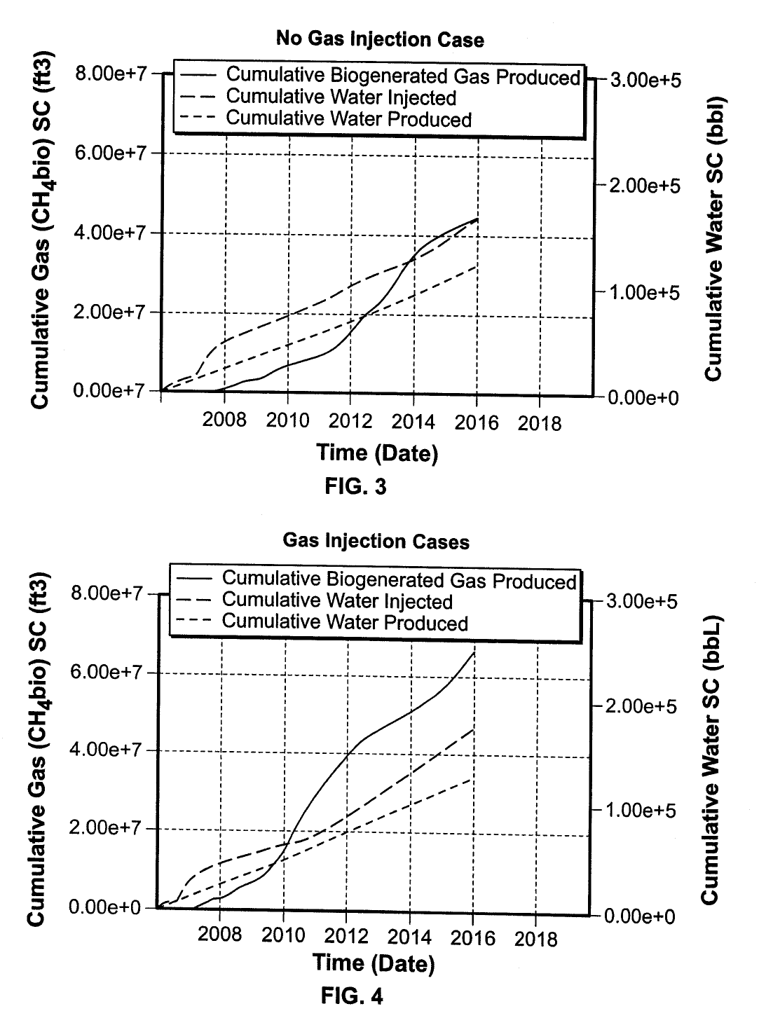

FIG. 3 is a plot of the cumulative volume of injected and produced water, and the cumulative biogenerated gas volume generated from the method described in this example. The total amount of biogenerated methane gas produced is 44.66 million cubic feet after ten years of production. The injected and produced volumes reported in the figure are at 1 atmosphere pressure and 15.5° Celsius.

### Example 1

### With Gas Injection

This example embodiment is identical to that of Example 0, except that gas is added to the injected fluids to help raise the reservoir gas saturation above the critical gas saturation. In the reservoir, when the gas saturation is lower that the critical gas saturation, the gas does not flow readily to the production well. In this example, not only the biogenerated gas adds to the gas saturation, but also injected gas helps maintain and raise the total gas saturation to reach the critical gas saturation earlier. FIG. 4 is a plot of the cumulative volume of injected and produced water, and the cumulative biogenerated gas volume. The total amount of biogenerated methane gas produced from the reservoir is 67.02 million cubic feet after ten years of production. A comparison between Examples 0 and 1 demonstrates that added gas injection helps to produce biogenerated gas from the reservoir by enhancing the gas saturation in the reservoir and supporting it so that it exceeds the critical gas saturation. The injected and produced volumes reported in the figure are at 1 atmosphere pressure and 15.5° Celsius.

### Example 2

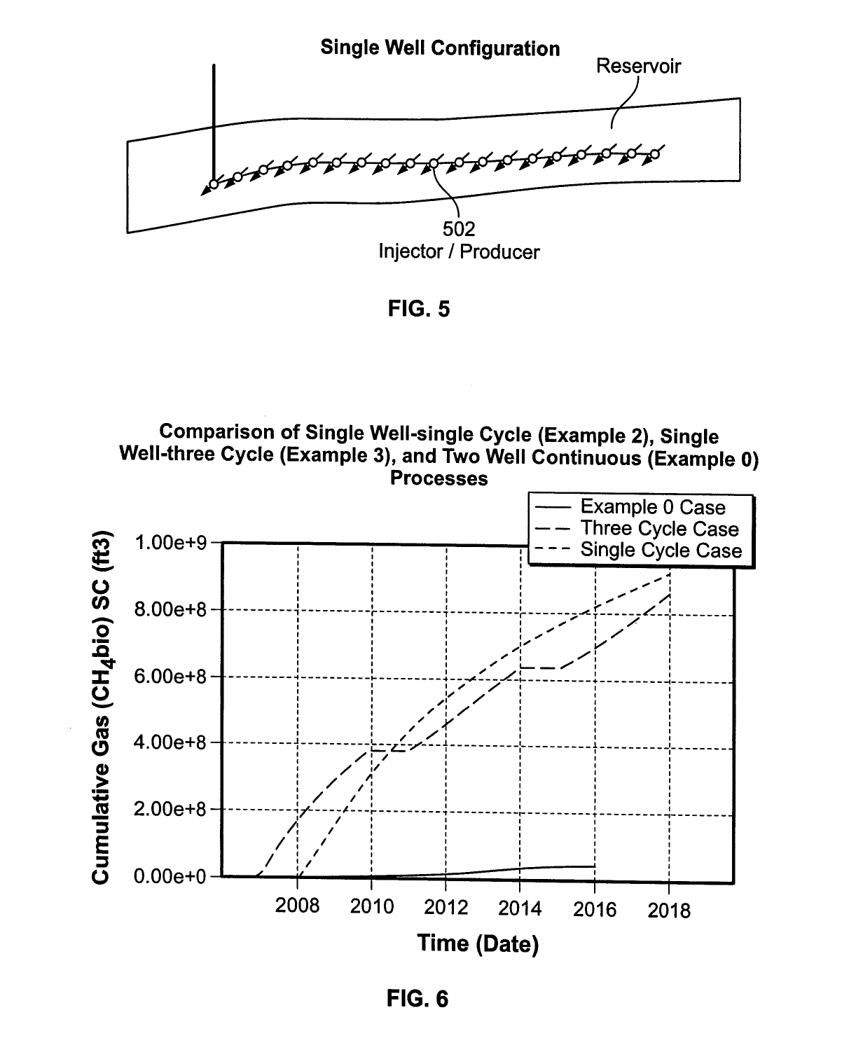

Data from a reservoir simulation model of a single well cyclic process carried out in a heavy oil reservoir is displayed in FIGS. 5 to 7. In this example, the process is single cycle and the horizontal well 502 shown in FIG. 5 is used as both an injection well and a production well.

The properties of carbon dioxide, hydrogen, methane, and water including molecular weight, critical pressure and temperature, heat capacity correlations, and viscosity correlations have been obtained from standard, publicly available databases. Standard diffusion and dispersion coefficients have been used. In the oil sands matrix, a standard set of oil-water and gas-liquid relative permeability curves, typical of that of heavy oil systems. The commercially available thermal reservoir simulation software package STARS™ was used for the reservoir simulations. In this example, methane-producing microbial consortia are present within the rock system.

In this example, the water/nutrient/gas mixture is injected into the reservoir through a horizontal well 502. The reservoir, wells, fluids, and microbial processes in the reservoir are modelled by using a reactive reservoir simulator. The microbial processes are represented by a set of chemical reactions with an accompanying set of reaction parameters and constants. The simulator solves for fluid velocities, pressure, fluid saturations, and component concentrations.

The length of the horizontal well is 1000 m. The permeability and porosity of the reservoir is 5D and 0.33, respectively. The initial oil and water saturations are equal to 0.5 each. The initial temperature and pressure of the reservoir is 10° Celsius and about 1590 kPa, respectively. At initial conditions, the reservoir system consists of an oil-water system so there is no free gas phase present. This can be an example of a post-water flooded reservoir.

In this example, the water plus nutrient plus gas is injected at 5000 kPa injection pressure for 2 years. The gas (in this case methane, but in other examples nitrogen, carbon dioxide or mixtures of non-condensable non-oxygen bearing gases) is added to the injection stream to raise the gas saturation in the reservoir. Non-oxygen bearing gases are preferred, although air can be used as an injected gas, e.g., if the volumes were such that all oxygen was consumed in the portion of the reservoir nearest the injection well reservoir by aerobic processes before encountering the methanogenic gas zones. On production, when the biologically-generated gas comes out of solution, the injected gas helps the total gas saturation (from bio-generated gas, solution gas, and injected gas) reach the critical gas saturation thus enabling free gas movement in the reservoir.

FIG. 6 displays the cumulative gas produced from the reservoir for the single cycle process of this example, three cycle process (see Example 3 below), and two well process described in Example 0. In this example, for the single cycle process, the cumulative biogenerated methane gas volume is 922 million cubic feet after ten years of production. The average methane gas rate during the production period, the production day methane gas rate (PDMGR), is about 252 thousand cubic feet per day. The injected and produced volumes reported in the figures are at 1 atmosphere pressure and 15.5° Celsius.

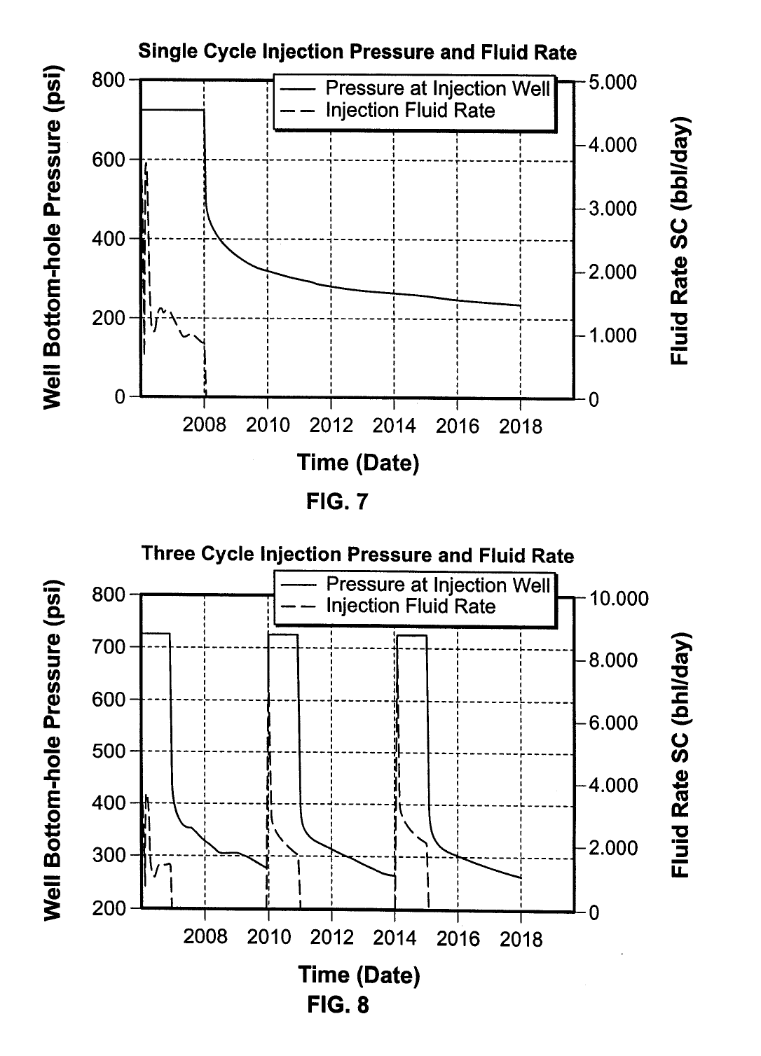

The horizontal well 502 is operated as both an injector and a producer. During the injection period, as shown in FIG. 7, water and nutrient and gas is injected into the reservoir at 725 psi (5000 kPa) injection pressure. The injection fluid rate is also plotted in FIG. 7. This injection pressure is the operating constraint on the injection well. During the injection period, the injected fluids mix with the reservoir fluids and the microbes start to consume the oil and water and generate methane and carbon dioxide.

After the injection period is done, the well is shut in and then remains inactive for a specified period of time. This period can be referred to as a soak period, where the injected fluids and reservoir fluids mix and the microbes consume the oil and water to produce methane and carbon dioxide and also increase reservoir pressure. The soak period is optional, that is, the well 502 can be converted directly from injection to production.

During the production period, the injection well 502 is converted to a production well and produces fluids from the reservoir. After the well starts to produce fluids, including biogenerated gas from the reservoir, the pressure in the reservoir falls and the fluid production rate declines. At some point, the fluid production rate falls below a target value and production from the well is stopped. For example, production from the well can be stopped if the gas production rate falls below a minimum economic rate of production.

After the well is taken off production, the well may be converted to an injection well and the injection, soak, and production periods are repeated. The cycles of injection, soak, and production can be repeated several times. In this example, with results shown in FIGS. 6 and 7, a single injection-soak-production cycle was conducted. The soak period in this example was 1 day during which the injection well was converted into a production well. Even though the gas recovery process was shut in after ten years, it could have been left on production for an extended time until a minimum specified gas rate was reached, after which the process could end or a new cycle could be started.

### Example 3

### Multiple Cycles

In another embodiment, multiple cycles of injection, soak, and production periods are done. FIGS. 6 and 8 display results of a three cycle process. As shown in FIG. 8, the nutrient/water/gas mixture was injected at 725 psi (5000 kPa) injection pressure. Each injection period of each cycle was 1 year long. For each cycle, the soak periods were 1 day and the production periods were 3 years long each.

As shown in FIG. 6, the cumulative biogenerated methane gas volume produced from the reservoir is 861 million cubic feet of gas after three cycles. In the first cycle, the production day methane gas rate (PDMGR) is 348 thousand cubic feet per day. In the second cycle, the PDMGR is 233 thousand cubic feet per day. In the second cycle, the PDMGR is 206 thousand cubic feet per day. Although three cycles were simulated in this example, additional production cycles could be done until the PDMGR dropped below a minimum economic value. The injected and produced volumes reported in the figures are at 1 atmosphere pressure and 15.5° Celsius. In this example, gas from the reservoir was produced after 1 year of water/nutrient/gas injection.

### Example 4

### Five-Spot Pattern

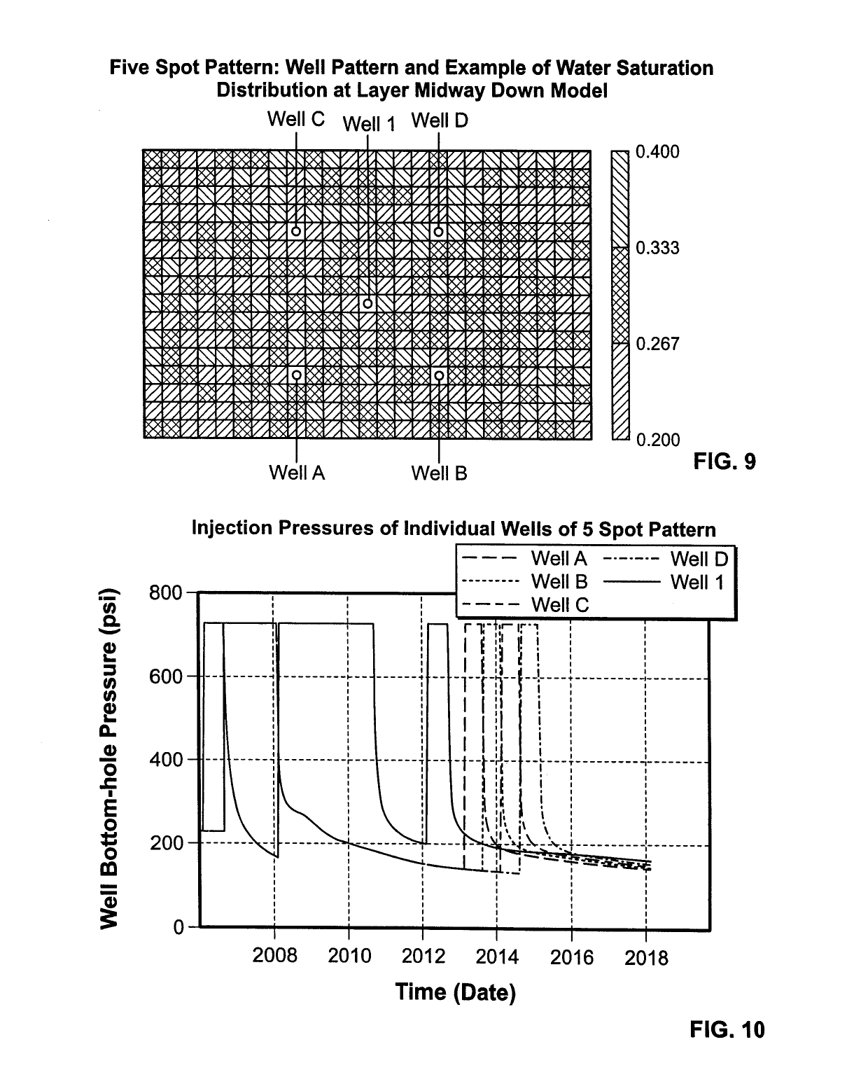

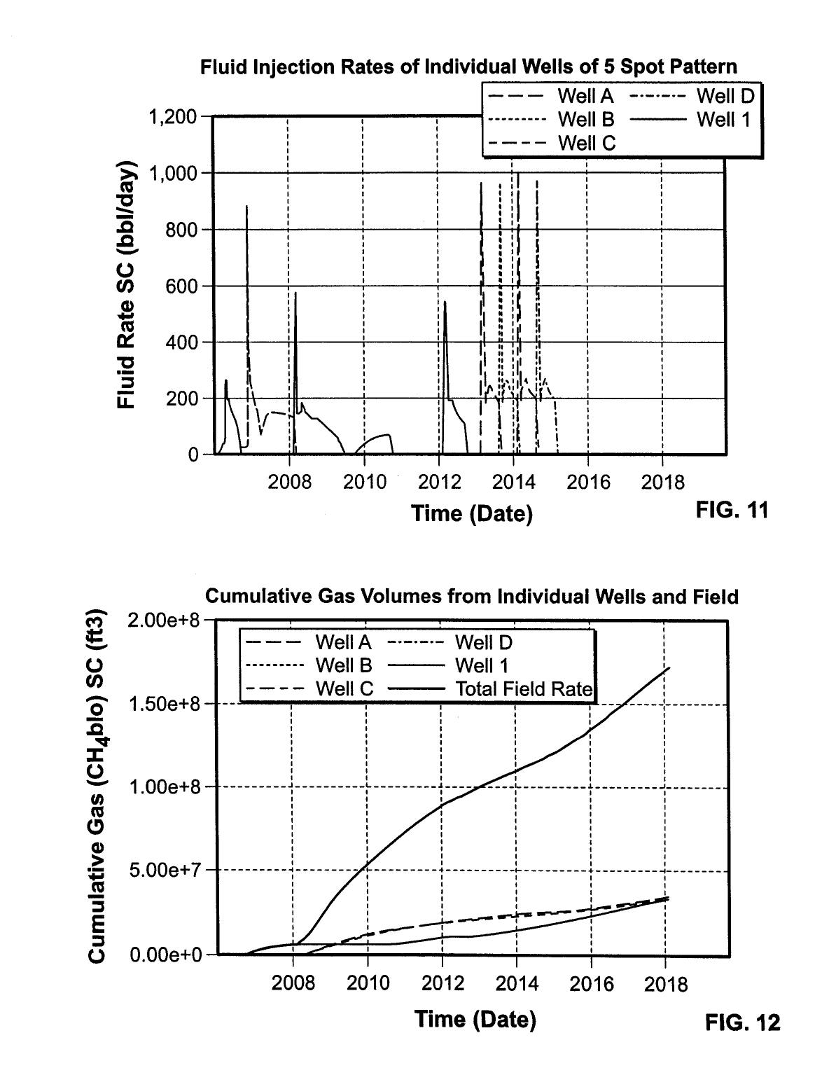

In this example, a five-spot pattern is used, as shown in FIG. 9. The reservoir thickness is 5 m, with permeability 5 D, and porosity 0.33. The initial average oil and water saturations are 0.712 and 0.288, respectively. The initial water saturation at one layer at mid-depth of the reservoir is displayed in FIG. 9. The initial temperature and average pressure of the reservoir is 10° Celsius and 1600 kPa, respectively. The wells shown in FIG. 9 are all vertical wells fully perforated across the reservoir thickness.

The process in this example is as follows. First, the nutrient/water/gas mix is injected into the centre well, well 1, at 725 psi (5000 kPa) injection pressure for 7 months, after which the centre well is converted to production and the remaining four outer wells, wells A, B, C, and D, start injection of the nutrient/water/gas mixture at 725 psi (5000 k Pa) injection pressure. After 17 months of fluid production from the centre well and mixture injection into the outer wells, the centre well is switched to mixture injection and the outer wells are switched to fluid production. After 31 months of injection into the centre well, it is converted to production. Seventeen months later, the centre well is converted back to injection. Seven months later, the centre well is converted once again to production.

From the start of the process, up to this point, the centre well has undergone three cycles. The outer wells are then converted briefly to injection in a rotating manner with first Well A for 6 months of injection, after which it is converted to production and then Well B has 6 months of injection, after which it is converted to production, then Well C with 6 months of injection, after which it is converted to production, and then Well D with 6 months of injection, after which it is converted to production. During this process, the outer wells experience two cycles. During injection, the pressure is at 725 psi (5000 k Pa). Thus this rotating action through the five-spot pattern yields fluid movement in the reservoir helping to mix nutrients with oil and water in the reservoir and create new oil-water interfaces to enable rapid microbial oil to methane conversion. The nutrient/water/gas mixture rates for the wells during injection periods are displayed in FIG. 11.

The cumulative biogenerated methane gas produced from the five-spot pattern is 172.1 million cubic feet over ten years of operation. This corresponds to an average production day methane gas rate (PDMGR) of 47.2 thousand cubic feet per day. The injected and produced volumes reported in the figures are at 1 atmosphere pressure and 15.5° Celsius.

In any of the examples described here, the well configurations can be vertical, deviated, horizontal, directionally-drilled, extended-reach, multilateral, and other well configurations. Also, the operating procedure for the field (a collection of wells) can have different groups of wells with different numbers of cycles or different injection, soak, or production periods. For example, a set of production wells (operating at relatively low pressure) may have a layer of soak wells between themselves and a set of injection wells (operating at relatively high pressure). This can be used to reduce or delay inter-well fluid communication. In other implementations, the layout of injection wells (high pressure) and production wells (low pressure) can be used to promote mixing in the reservoir.

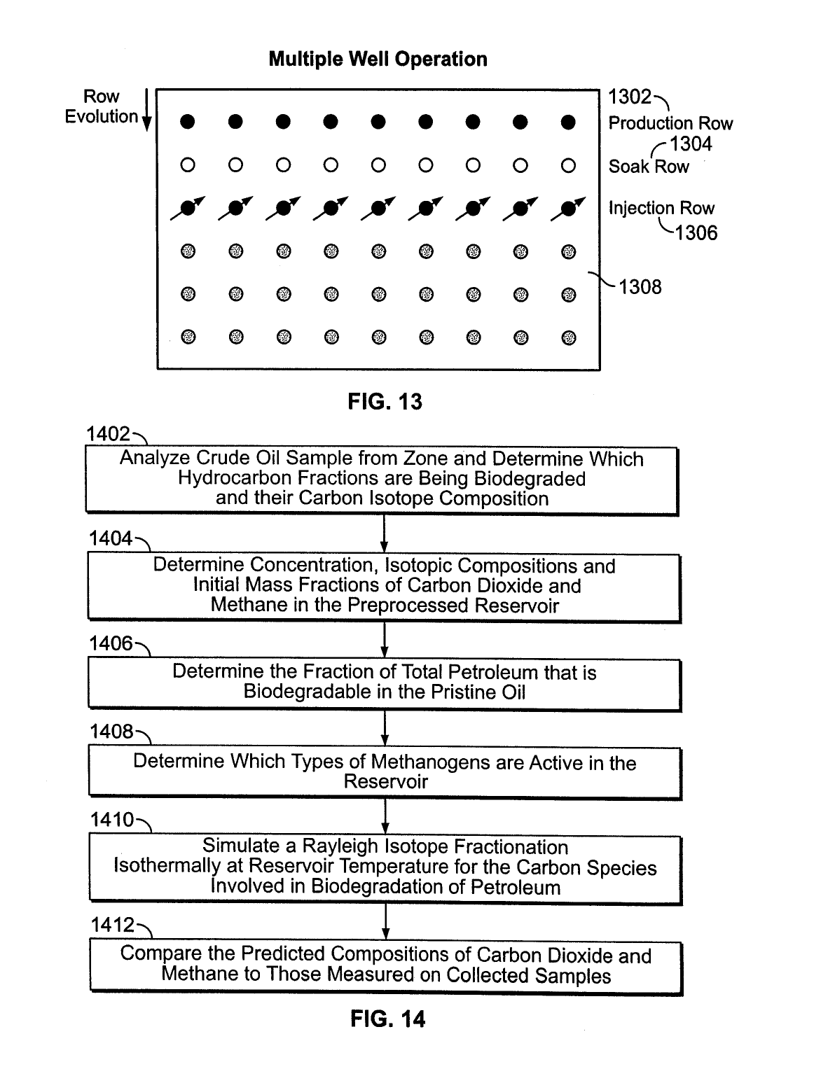

An example is shown in FIG. 13. In this example, after the injection row 1306 has injected a desired target volume of nutrient/water/gas mixture, the wells in the row 1306 are switched to soak. After a specified amount of time, for example the time it takes to build the gas saturation in the reservoir near the soaking wells to the critical gas saturation, the wells are converted to production. The wells upstream of the now soaking wells, e.g., the wells in row 1308, are then converted to injection. Thus, injection of the nutrient/water/gas mixture moves down the field in a row-wise manner with a soaking row 1304 directly behind the injection row 1306, and production row directly behind a soaking row. After the economic methane gas rate or other termination criterion has been reached in the production row 1302, then the production row 1302 can be placed back on injection and the sequence can continue through the reservoir.

In some implementations, conversion from production to injection, or termination of the process as a whole, can be controlled by a minimum methane gas production rate, called the abandonment methane gas rate. The gas flow rate from the reservoir is affected by the reservoir pressure. As depletion of reservoir fluids occurs and the fluids are produced to the surface, the pressure in the reservoir falls and thus the driving force responsible for pushing fluids to the surface declines.

In any of the examples described here, the fluids production rate can be slowed down so that the decline of the pressure in the reservoir as a well goes on production is reduced. Methods and systems for controlling operating parameters when producing the biogenerated gas are discussed in further detail below, to extend the production of the gas from the reservoir.

### Monitoring During Production

While gas is being produced from the zone, including the biogenerated methane, production parameters including the reservoir pressure and the composition of the produced gas can be monitored. The pressure can be monitored using one or more pressure monitoring devices. For example, continuous pressure measurement devices, such as pressure transducers or piezometers, can be mounted in the wells and can provide real-time, periodic and/or on-demand measurement data.

Monitoring the composition of the produced gas includes monitoring the isotopic composition of the produced gas. In some implementations, the carbon isotopic compositions of carbon dioxide and/or methane included in the produced gas can be analyzed. By way of other examples, the hydrogen isotopic composition of methane and/or hydrogen in the produced gas can be analyzed, and/or the oxygen isotopic composition of carbon dioxide in the produced gas can be analyzed. In some implementations, the hydrogen and/or oxygen isotopic composition in produced water can be analyzed. In other implementations, carbon or hydrogen isotopic composition of produced oil components can be monitored and analyzed. In some implementations, the monitored production parameters can include the flow rates of the gas, oil and/or water being produced.

When the process is operating optimally at peak gas generation, the reservoir pressure can be constant or increasing during gas production and the gas flow rate in the production well is constant or increasing. Away from peak gas generation, reservoir pressure may decrease during gas production, but the process can continue. Although gas and fluid is being withdrawn from the reservoir, which has the effect of lowering the reservoir pressure, methane is also being generated at the same time, which has the effect of raising the reservoir pressure. A pressure drop can indicate a change in the zone's gas saturation. As discussed above, preferably the zone's gas saturation is at least the critical gas saturation during gas production. Accordingly, monitoring the reservoir pressure can help indicate when this is not the case.

The composition of the produced gas can be measured by analyzing samples of the produced gas and measuring the proportions of the various gases included therein, e.g., methane, carbon dioxide and hydrogen. Generally, the produced gas will include one or more of the following: (1) solution gas; (2) biogenerated methane and gaseous byproducts; and (3) injected gas. Solution gas is gas that was initially present in the zone before modifiers and fluids were injected. Solution gas typically includes methane, carbon dioxide, nitrogen, and small amounts of ethane and higher alkanes. The biogenerated methane is methane that is generated as a result of stimulating the microbial conversion of petroleum in the zone to methane. The gaseous byproducts include gases that can also be produced by the reactions generating the methane or during intermediate stages of the conversion process, and include carbon dioxide and hydrogen. The injected gas is the gas that was injected during the injection phase, for example, carbon dioxide, nitrogen and/or methane.

The proportions of methane and carbon dioxide produced if the process is operating properly can be predicted using a balanced chemical equation. For example, the proportions of methane and carbon dioxide produced if the process is operating properly and n-alkanes are being converted, can be in the ratio predicted by the following stoichiometric equation:

$${C}_{n} {H}_{2 n + 2} + \frac{( n - 1 )}{2} {H}_{2} O \to \frac{( n - 1 )}{4} {CO}_{2} + \frac{( 3 n - 1 )}{4} {CH}_{4}$$

The term CH₄ₜₕₑᵣₘₒ herein refers to background thermogenic methane originally in the reservoir. The CH₄ₜₕₑᵣₘₒ may have been contaminated with some biologically generated methane produced locally by biological activity, e.g., in shales encasing the reservoir. A typical δ¹³C CH₄ₜₕₑᵣₘₒ can be approximately −45 per mil PDB (PeeDee Belemnite standard used as a δ¹³C). The term CH_{4bio} refers to the biologically produced methane generated in the reservoir using the acceleration techniques, such as those described above. The CH_{4bio} may have a generated δ¹³C CH_{4bio} of a wide range, e.g., from approximately −70 per mil to as low as −35 per mil. The term CO_{2bio} refers to the carbon dioxide measured in the produced gas, and can have a range of δ¹³C from approximately −30 per mil to +20 per mil.

The ratio of CH_{4bio} to CH₄ₜₕₑᵣₘₒ can be assessed isotopically through carbon isotope analysis of methane collected from the reservoir to measure the effectiveness of the biogenerated methane generation in situ and production to surface. The degree of conversion of petroleum to methane in the reservoir can be assessed using the carbon and/or hydrogen isotopic composition of methane, carbon dioxide and/or hydrogen collected from the reservoir.

In some implementations, samples of produced fluid, including gas, oil and water, can be taken at intervals ranging from days to weeks. The fluid can be prepared for isotopic analysis using isotope ratio mass spectrometry. As an example, carbon and hydrogen isotopic analyses of produced gas can be performed using isotope ratio mass spectrometry using, for example, on line gas chromatography combined with isotope ratio mass spectrometry (GCIRMS). Such a system can be conveniently located near a production well site to provide real time information that can be used for real time control of the injection and/or production processes. In other implementations, the system can be located offsite.

In one implementation, the process of methanogenic biodegradation can be assessed isotopically and by using bulk gas composition (concentrations and proportions of carbon dioxide and methane and sometimes hydrogen) using a model as described below, which can be coupled to a reservoir simulator to predict bulk and isotopic gas composition. Coupling the model to a reservoir simulator can allow the use of pressure, produced fluid distribution and quantity, and the bulk and isotopic chemistry of produced gases from gas, water or oil production to control processes in the reservoir. In one implementation, a Rayleigh isotope fractionation model can be used to assess the extent of conversion of petroleum to methane, and partition of carbon from the biodegradation process between CH₄ and CO₂. In some implementations, isotopic tracers, e.g., deuterium labeled hydrogen, carbon or oxygen isotopically labeled carbon dioxide, or carbon or hydrogen labeled methane, can be added to the reservoir as injected pulses (e.g., can be included in the injected fluid or injected modifiers) to assess activity of methanogens and adverse organisms, such as hydrogen oxidizers (sulphate reducing organisms, iron reducing organisms and/or methanotrophs), which can affect competing processes to methanogenesis.

Referring to FIG. 14, a flowchart shows an example method 1400 for using the Rayleigh isotope fractionation model to assess the process of methanogenic biodegradation. A crude oil sample from the zone is retrieved and analyzed to determine which hydrocarbon fractions are being biodegraded (Step 1402). For example, this can be done by geochemically comparing the oil sample to an equivalent less biodegraded oil from another well, or by examining the general changes in oil composition in various oils from the field of interest. Geochemical comparisons can involve quantitation of specific saturated and aromatic hydrocarbons found in crude oils, that are typically consumed during biodegradation. Additionally, sometimes the measurement of the carbon isotopic composition of these same hydrocarbons can be used. The analysis can also include determining the bulk carbon and hydrogen isotopic composition of the hydrocarbon fractions or specific molecular components in the oils.

The concentration, isotopic compositions and initial mass fractions of carbon dioxide and methane in the original preprocessed reservoir are determined from collected gas samples (e.g., produced gas) (Step 1404). For example, most Lower Cretaceous oils in the Alberta basin have a bulk δ¹³Cₒᵢₗ value of −29 to −31% and typical thermogenic gas associated with non-degraded oils has δ¹³C_{cH4} values of −42 to −48%, and a δ¹³C_{co2} value of −3 to −19%. The fraction of the total petroleum of the pristine oil (with δ¹³C of −29%) that is degradable is determined (Step 1406). For example, the fraction of the total petroleum that is degradable can be measured using chromatographic procedures to determine the components that are degrading and the mass fraction of the oil they represent. These analyses can also be supported by laboratory biodegradation experiments (Step 1406). A microbiological laboratory analysis is carried out to determine which types of methanogens (e.g., acetoclastic or hydrogen reducing or hydrogenotrophic) are active in the reservoir (Step 1408) and in which proportions.

The data obtained from Steps 1404 to 1408 can be used as input to perform a Rayleigh isotope fractionation calculation. The Rayleigh isotope fractionation calculation can be simulated isothermally at reservoir temperature for the carbon species involved in biodegradation of petroleum via syntrophic alkane oxidation, syntrophic acetate oxidation, acetoclastic methanogenesis and hydrogenotrophic methanogenesis (Step 1410). The predicted bulk compositions, quantities and isotopic compositions of carbon dioxide and methane are compared with those measured from collected samples (Step 1412).

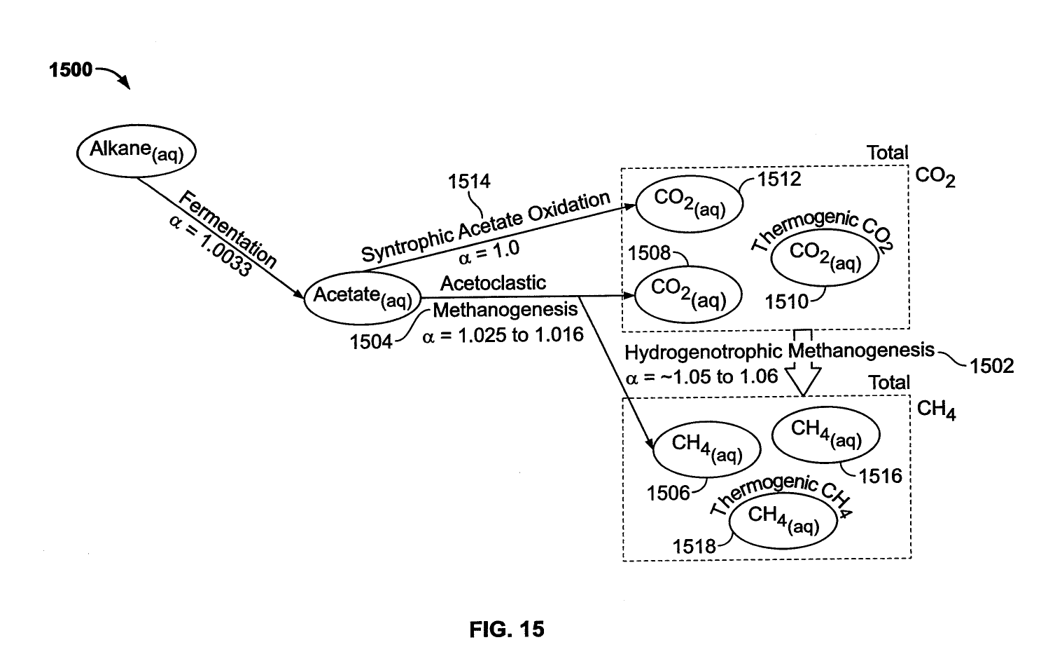

A box diagram representing the conceptual model of the Rayleigh isotope fractionation simulation for carbon isotopes in the system is shown in FIG. 15. The box diagram is representative of microbiological processes occurring in the biodegradation reactive zone in the reservoir where dissolved petroleum compounds in an aqueous phase are metabolized by a consortium of microorganisms that produce CO₂ and CH₄ as terminal species. There is no isotopic fractionation between methyl and carboxyl carbon in the acetate produced, as it is derived from a wide range of precursors and substantially all the acetate is converted to carbon dioxide and methane, which is a reasonable assumption given the low concentrations of acetate associated with biodegraded petroleum reservoirs. Species in the Rayleigh isotope fractionation box diagram are assumed to be in an aqueous phase as dissolved components, where they are most available to microorganisms. Alpha (α) is the fractionation factor for each reaction, which describes the relative rates of reaction of sites with ¹²C as opposed to ¹³C present. The α values can be determined in laboratory experiments in which reservoir microorganisms convert hydrocarbons of known isotopic composition to methane and carbon dioxide under conditions similar to reservoir conditions, and in which the isotopic composition of the products is determined, e.g., by isotopic ratio mass spectrometry.

The Rayleigh isotope fractionation simulation operates by numerically converting all biodegraded hydrocarbons to carbon dioxide portioning the resulting carbon dioxide between pools destined for hydrogenotrophic methanogenesis 1502 and acetoclastic methanogenesis 1504. Half of the CO₂ in the acetoclastic reaction pool is converted to CH₄ 1506 with an appropriate temperature controlled isotope fractionation factor. Carbon dioxide 1508 destined for hydrogenotropic methanogenesis, along with CO₂ₜₕₑᵣₘ 1510 and with CO₂ 1512 generated from syntropic acetate oxidation 1514 is converted to CH₄ 1516 by hydrogenotropic methanogenesis. The total carbon pool is converted to methane and carbon dioxide to various degrees through the two methanogenic pathways, and is admixed with the existing carbon dioxide and methane in the reservoir, to calculate the concentrations and isotopic compositions of the resultant carbon dioxide and methane in the reservoir. It is assumed there is no source of carbon dioxide other than from alkane degradation. Isotopic fractionation of carbon species during phase changes (e.g., partition between gas and liquid) is small and is considered negligible. The total resulting methane includes CH₄ 1506 generated from acetoclastic methanogenesis, CH₄ 1516 generated from hydrogenotropic methanogenesis and admixed thermogenic methane CH₄ₜₕₑᵣₘ 1518.

Isotope fractionation factors can be important to simulating the methanogenesis processes. For example, fractionation factors for acetoclastic and hydrogenotrophic methanogenesis can be collated from available literature, and cover a range of methanogenic taxa, including mesophiles and thermophiles. There is a general decrease in fractionation factor with increasing temperature, which is consistent with isotope systematics theory. On the whole, the acetoclastic methanogens tend to isotopically fractionate carbon much less than the hydrogenotrophic methanogens. The fractionation factors are related to specific microorganisms and also to the availability of hydrogen, which may be a greater factor in fractionation than the temperature. The inventors have noted an apparent linear trend of fractionation factor with temperature. From field samples, laboratory cultures can be used to establish for the actual microorganisms from a particular reservoir, the effective isotopic fractionation factors suitable for simulating the isotopic evolution of gases in the specific reservoir being processed.

The temperature-dependent relationships for the fractionation factors for each methanogenesis reaction, the reservoir temperature and the percent of CO₂ reduced via hydrogenotrophic methanogenesis (as opposed to acetoclastic methanogenesis) are also needed to determine a net fractionation factor and can be determined by microbiological analysis of the reservoir microorganisms using quantitative PCR (polymerase chain reaction) analysis or other microbiological techniques including 16sRNA gene fingerprinting to determine species distributions. The temperature dependency of the isotope fractionation factors can be determined by controlled experiments. Isotopically labeled substrates can be fed to cultures of reservoir organisms at different temperatures under conditions, whereby the isotopic fractionations of different components can be monitored by separation of the products, such as carbon dioxide and methane, and subjecting them to isotopic analysis. The hydrogen isotopic compositions of methane and any produced hydrogen can also be assessed and can be used to monitor the progress of the petroleum to gas conversion.

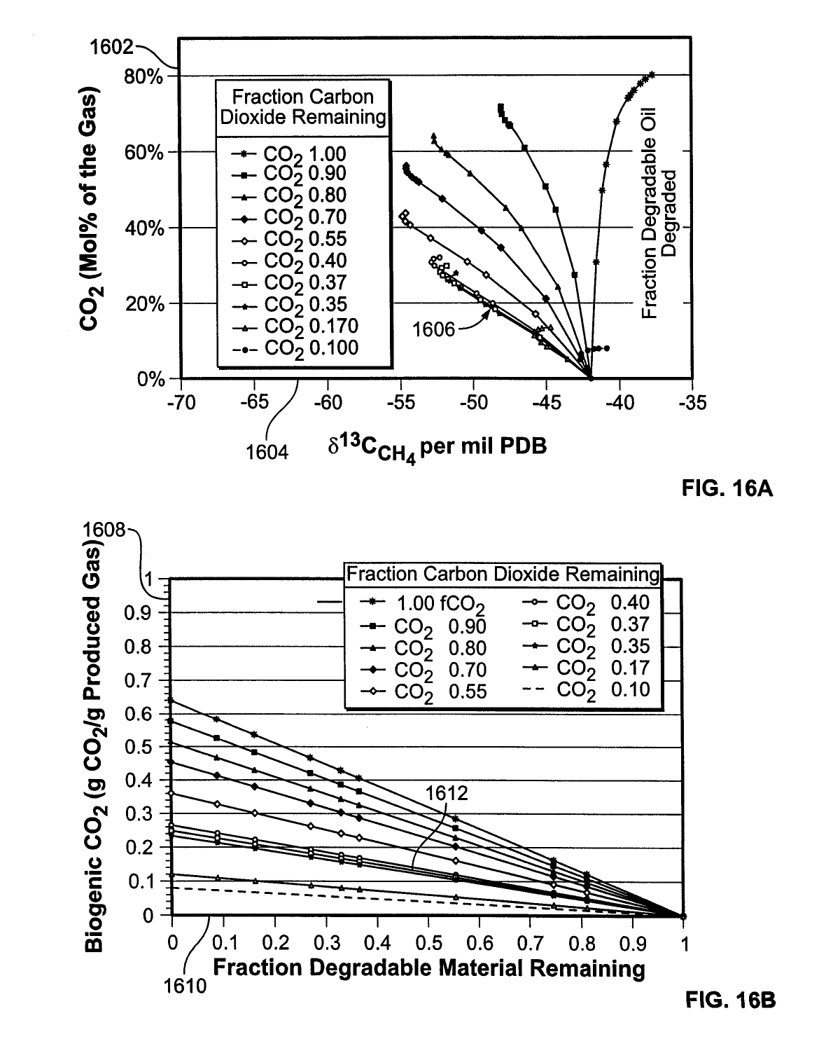

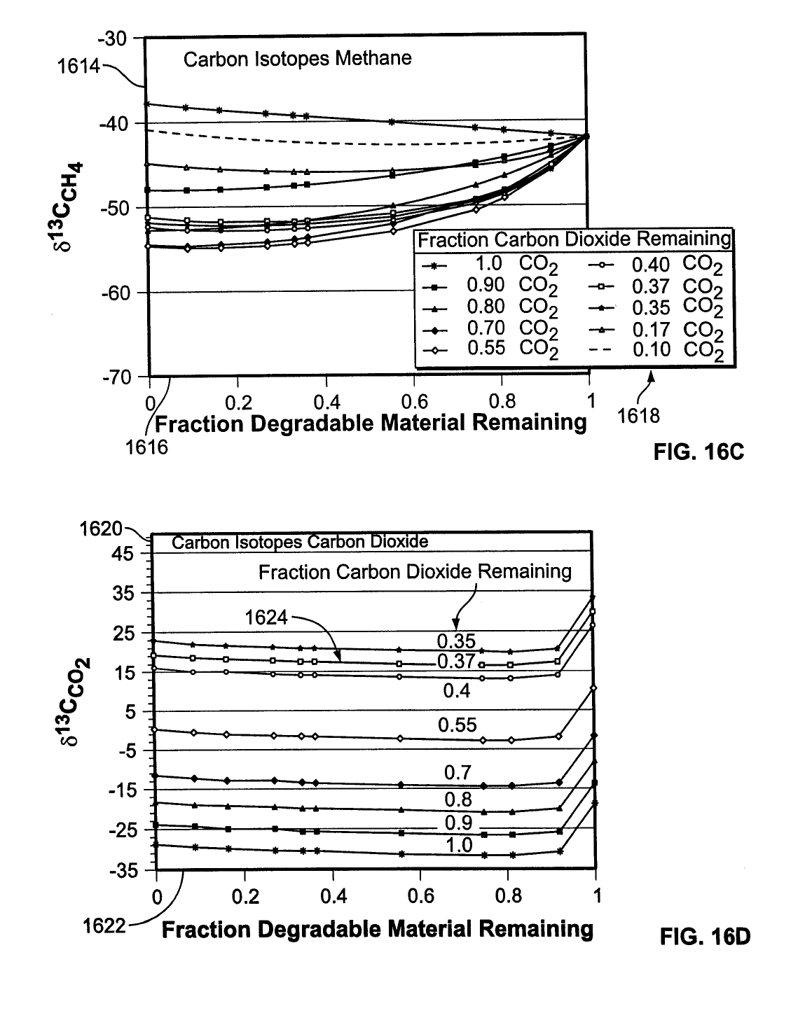

During gas production from the reservoir, in addition to monitoring fluid flow rates, compositions (oil, water, gas) and reservoir pressures, oil, water and gas samples can be collected from observation or production wells from time to time and analyzed for molecular and carbon, hydrogen and oxygen isotopic composition. Plots for the carbon isotopic compositions followed by carbon dioxide and methane in a reservoir that is being processed can be produced by simulation of the process shown in FIG. 15, e.g., using software, and the values of isotopic fractionation factors and initial oil and gas compositions described above. Example plots for illustrative purposes are shown in FIGS. 16A-D. The plots describe the composition of carbon dioxide and methane in terms of both the quantities of the gases and the carbon isotopic evolution of the components as the reactions of the conversion process proceed.

In the particular example used to generate these illustrative plots, the reservoir was a typical Western Canadian heavy oil reservoir from the Lloydminster area. Table 1 below shows a set of example parameters used for a control process calculation. A control process calculation can be used to establish whether the microbial conversion process is proceeding correctly by analysis of produced gases together with monitoring well flow rates and pressures, as shall be described further below. Referring again to the steps in FIG. 14, the input parameters for the control process calculation include:
initial pre-process methane and carbon dioxide contents of the in situ live oil (petroleum) in the reservoir (see Step 1404);
the type of compounds in the petroleum being biodegraded and their carbon to hydrogen ratio (see Step 1402);
the molecular weight of the degrading petroleum fraction;
the initial carbon isotopic composition of the carbon dioxide and methane in the reservoir (see Step 1404); and
the initial carbon isotopic composition of the hydrocarbons being degraded (see Step 1402);
the proportion of hydrogenotrophic and acetoclastic methanogens active in the reservoir (see Step 1408); and
the carbon isotopic fractionation factors for the microbial alkane oxidation (αₐₗₖₐₙₑ) and net methanogenesis processes (αₘₑₜₕₐₙₑ) as a function of temperature.

TABLE 1

| Input Parameter | Value (Example) |
|---|---|
| Initial CH₄ g/g Petroleum | 0.05 |
| Initial Degradable Material g/g Petroleum | 0.2 |
| Z Number Degradable Compound Type | 1.8 |
| Initial CO₂ g/g Petroleum | 0.001 |
| Initial δ¹³C_{CO2} | −19 per mil |
| Initial δ¹³C_{CH4} | −42 per mil |
| % H₂/CO₂ Methanogens | 85% |
| % Acetoclastic Methanogens | 15% |
| Alkane δₒ | −28 per mil |
| αₐₗₖₐₙₑ | 1.0033 |
| αₘₑₜₕₐₙₑ | 1.0517 |
| MW Petroleum | 200 g/mol |
| Reservoir Temperature | 25° Celsius |

In the above example, the compounds in the petroleum being biodegraded are alkanes with an average molecular formula of CₙH_{1.8n}. The above values shown in Table 1 can be determined by laboratory analysis of the initial petroleum in the reservoir, microbiological analysis of the reservoir pre-process or of water samples during the production operation, and from laboratory studies at reservoir conditions and/or from published data.

Once the initial reservoir conditions are described chemically and microbiologically, the composition of produced gases and in particular, the carbon and hydrogen isotopic composition of the methane and carbon dioxide in the produced gases can be used to track the process operation and suggest modifications to the operating process. While carbon isotopic variations are illustrated here, the hydrogen isotopic compositions of methane and produced waters can also be used to assess and monitor the process. The oxygen isotopic composition of produced water and carbon dioxide can also provide information on the correct operation of the process and the molecular and isotopic compositions of produced oil hydrocarbon and nonhydrocarbon components can also be used to monitor and assess the process operation. Injection of carbon or hydrogen isotopically labeled alkanes, carbon labeled CO₂ or acetate, or deuterium labeled hydrogen gas by injection to a well, can also be used to trace the correct operation of the process by analysis of the isotopic composition of produced carbon dioxide, methane and water. The bulk and isotopic composition of the produced gases, e.g., the methane and carbon dioxide, together with reservoir pressure and well flow rate measurements as well as analyses of any waters or oils produced and microorganisms in the waters can then be used to assess whether the reaction system is operating optimally and to indicate appropriate changes in operating parameters to improve process efficiency. An example is illustrated below.

Referring again to FIGS. 16A-D, input parameters, e.g., those shown in Table 1, can be input into the process control model to predict for a properly operating process particular quantities and isotopic compositions of carbon dioxide and methane, which can then be compared to the actual quantities and isotopic compositions measured in gases during the production process. In the example plots shown, the following are predicted from the Rayleigh isotope fractionation model: the carbon and hydrogen isotopic composition of methane; the carbon isotopic composition of carbon dioxide; and the methane and carbon dioxide contents of produced gases. Reservoir pressure and fluid flow rates in production wells can also be predicted by coupling the isotope modeling software to a reservoir simulator such as CMG's STARS™ program. Produced water and gas chemistry can be examined to detect traces of indicators, such as hydrogen content, water soluble sulphides or hydrogen sulphide content, sulphate, nitrate or other undesirable electron acceptors (waters), or for the presence of adverse microorganisms such as sulphate reducing bacteria or methanotrophic archaea.

FIG. 16A shows a plot of CO₂ content of produced gas (vertical axis 1602) versus isotopic composition of methane (horizontal axis 1604). The lines represent various conversion efficiencies of carbon dioxide to methane, tracking the carbon dioxide remaining unconverted (1=100% unconverted and so on). A preferred process operation line is indicated by the heavier line 1606 which represents a fraction of carbon dioxide remaining of 0.37 (37 wt % CO₂ remains after the process) fraction carbon dioxide remaining.

FIG. 16B shows a plot of CO₂ content of produced gas (vertical axis 1608) versus fraction of carbon dioxide converted (horizontal axis 1610). The lines represent various conversion efficiencies of carbon dioxide to methane tracking the carbon dioxide remaining unconverted (1=100% unconverted and so on). A preferred process trajectory is indicated by the line 1612.

FIG. 16C shows a plot of carbon isotopic composition of methane (vertical axis 1614) versus fraction of degradable oil converted (horizontal axis 1616). The lines represent various conversion efficiencies of carbon dioxide to methane, tracking the carbon dioxide remaining unconverted (1-100% unconverted and so on). A preferred process trajectory is indicated by the line 1618, which represents 0.37 fraction carbon dioxide remaining (i.e., 37 wt % CO₂ remains in the reservoir).

FIG. 16D shows a plot of carbon isotopic composition of carbon dioxide (vertical axis 1620) versus fraction of degradable oil converted (horizontal axis 1622). The lines represent various conversion efficiencies of carbon dioxide to methane, tracking the carbon dioxide remaining unconverted (1=100% unconverted and so on). A preferred process trajectory is indicated by the line 1624.

### Controlling the Operating Parameters

Based on the monitored production parameters, at least one of either stimulation of the microbial conversion or the gas saturation in the zone is adjusted, to control the composition of the produced gas or the mobility of the biogenerated methane respectively. The monitored production parameters include the reservoir pressure and the isotopic composition of at least one of either methane or carbon dioxide or hydrogen included in the produced gas. Adjusting the stimulation of the microbial conversion and/or the gas saturation can enhance the production of the biogenerated methane from the zone to surface.

In other implementations, without being limiting, the monitored production parameters can include one or more of the following: the gas production flow rate, the water production flow rate, the oil production flow rate; the composition of the produced water; the composition of the produced oil; or the concentrations of one or more types of gas in the produced gas.

As discussed above, the reservoir pressure can be used to estimate the zone's gas saturation. Preferably, the zone's gas saturation is greater than or equal to the zone's critical gas saturation, so that the gas phase in the zone is mobile and can be produced as free gas. The reservoir pressure is therefore monitored to estimate the gas saturation. The reservoir pressure, and therefore the gas saturation, can be adjusted by controlling one or more operating parameters. Operating parameters refers to parameters that can affect the injection into or the production out of the reservoir.

If the reservoir pressure decreases and the gas production flow rate increases (or is relatively high), then more carbon dioxide and less methane may be produced. In some circumstances, reducing the gas production flow rate can increase the methane content of the produced gas. A balance can be found between the reservoir pressure and the gas production flow rate to enhance the production of the biogenerated gas (rather than other gases) from the reservoir. The following is a list of example operating parameters that can be adjusted to control the reservoir pressure and to find a balance between the reservoir pressure and the gas production flow rate:
injector well flow rate;
production well flow rate;
composition of injected modifiers;
quantity of injected modifiers;
composition of injected purge gas;
quantity of injected purge gas;
quantity of injected water;
frequency of injecting purge gas;
injection, soak and production time tables.

The list is not exhaustive, and other operating parameters can be adjusted. Two or more operating parameters can be adjusted simultaneously, in addition to adjusting single operating parameters.

The reservoir pressure can be increased, in one example, by increasing the volume of biogenerated methane in the zone. The volume of methane being microbially converted from petroleum within the zone can be controlled to some degree by adjusting operating parameters including, for example: adjusting the composition and/or quantity of the modifiers injected into the reservoir and the production well valve opening. The reservoir pressure can also be increased by increasing the volume of fluid injected into the zone, for example, by adjusting the injector well flow rate, the quantity of injected purge gas and frequency of injecting purge gas.

Determining what operating parameters to adjust and by how much so as to adjust the stimulation of the microbial conversion and/or the gas saturation, can be determined based, at least in part, on the isotopic compositional analysis of the produced gas and the reservoir pressure. In some implementations, the interaction of produced gas composition, production well flow rate and the reservoir pressure can be used to determine what operating parameters to adjust.

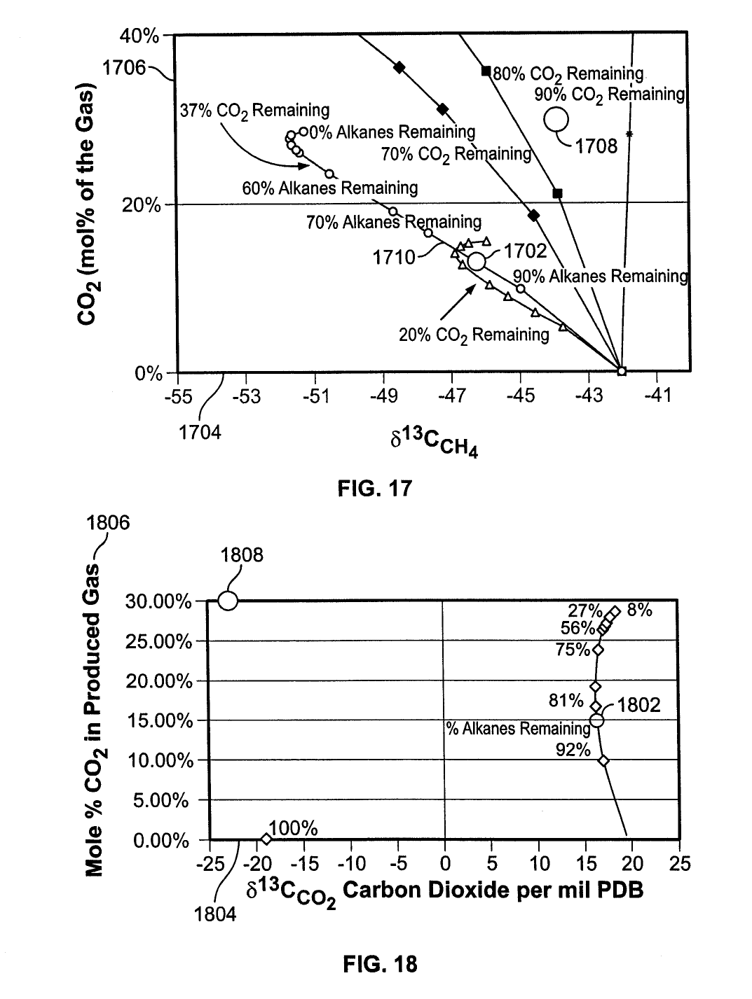

Referring now to FIGS. 17 and 18, an illustrative example based on a reservoir simulation shall be discussed. The example demonstrates how the reservoir pressure, gas production flow rate and composition of the produced gas and water, including the isotopic composition of at least one component (e.g., methane or carbon dioxide) included in the produced gas, can be used to adjust at least one of the stimulation of the microbial conversion or the gas saturation to enhance production of the biogenerated methane from the reservoir.

In this particular example, a cyclic version of the process is implemented in a Lloydminster area Sparky Formation reservoir. A cyclic version refers to two or more cycles of injection and production, which may or may not include a soak period in between. The control modeling process used the data in Table 1 above.

Referring particularly to FIG. 17, a gas composition for two cycles of one embodiment of the process is shown. FIG. 17 is an enlarged view of the plot shown in FIG. 16A. The x-axis 1704 is the carbon isotopic composition of the methane produced at the production well and the y-axis 1706 is the mole % of carbon dioxide in the produced gases at the production well, the remainder being mostly methane. After cycle 2, the produced gas has the composition shown in the circle 1702, and the reservoir pressure is stable at 4500 kPa, with a well gas flow rate of 300 Mcf/day, which has been maintained for 3 days. After cycle 4, the produced gas has the composition shown in the larger circle 1708, the reservoir pressure is 3850 kPa and the gas flow rate is 130 Mcf/day, which flow rate is declining rapidly. The lines indicate the gas composition trajectories expected for various proportions of carbon dioxide reduction to methane. The line 1710 at 37% CO₂ remaining is the line on which a properly operating process lies with optimal conversion of carbon dioxide to methane for this system. The points on the line indicate what fraction of the biodegradable hydrocarbons has been converted to carbon dioxide and methane. In normal operation, the gas compositions move along the line as more petroleum is converted into gas.

Referring now to FIG. 18, a gas composition for the same two cycles of one embodiment of the process as discussed above. The x-axis 1804 is the carbon isotopic composition of the carbon dioxide produced and the y-axis 1806 is the mole % of carbon dioxide in the produced gases. After cycle 2, the produced gas has the composition shown in the circle 1802. After cycle 4, the produced gas has the composition shown in the larger circle 1804. The lines indicate the gas composition trajectories expected for various proportions of carbon dioxide reduction to methane if the process is operating correctly. The points on the line indicate what fraction of the biodegradable hydrocarbons (in this case n-alkanes) have been converted to carbon dioxide and methane.

The two figures indicate that at cycle 2 the gas compositions lie on the 37 wt % remaining fraction of generated carbon dioxide line (the other 63 wt % of the generated gas being largely methane). The gas compositions indicate the process is operating well and gas production rates of 300 Mcf/day and a stable reservoir pressure indicates the process is in the optimum operating range, and that reservoir pressure can be reduced further to increase gas production rate if need be. Methanogenesis can operate at other conversion efficiencies of carbon dioxide to methane, however the optimal conversion is shown by the marked line 1710 in FIG. 17. If the process is operating at lower efficiencies of conversion of carbon dioxide to methane, so that more than 37% of the produced carbon dioxide is retained in the reservoir, then this indicates that the nutrient composition is not ideal for the process or that adverse organisms are operative. Sampling water and making chemical analyses of components present and performing microbiological analyses on produced microorganisms found in production fluids can be used to suggest a remedy.

The gas composition (see FIG. 18) also indicates that approximately 85 wt % of the degradable alkanes are still present in the oil in the reservoir and this matches the simulation values generated by the simulator. Accordingly, the process is operating ideally and as planned.

FIGS. 17 and 18 also show the gas composition from cycle 4 of the process (1708 1808). At cycle 4, the gas composition has strayed away from the preferred process line, with too much carbon dioxide in the produced gases, isotopic compositions of methane and carbon dioxide are well away from the preferred values and traces of hydrogen sulphide are detected in the gas stream. The reservoir pressure has dropped to 3850 kPa and is still declining, with a gas flow rate of 130 Mcf/day, despite injection of water bearing nutrients. In this example, analysis of a produced water sample indicates that small amounts of sulphate ion have entered the reservoir. A microbiological analysis indicates sulphate reducing bacteria are present in the reservoir and microbial sulphate reduction activity has been occurring, making the gas sour and reducing methanogenesis. Accordingly, in response to this analysis, production cycle 4 is stopped and a new injection cycle is started with a nutrient injection fluid amended with sodium molybdate to inhibit sulphate reduction activity. Injection continues until the reservoir pressure reaches 4100 kPa. After a 14 day soak period, critical gas saturation levels are reached again, production is restarted and produced gases are methane rich with no hydrogen sulphide present. The gas bulk and isotopic composition has returned to values closer to the preferred process line.

This is just one example of the use of carbon and hydrogen isotopic composition of produced gases along with water chemistry and reservoir pressure assessment to monitor and control process operation.

In other implementations, active testing of the process also can be achieved with pulses of isotopically labeled carbon dioxide, methane or hydrogen or isotopically labelled acetate added to the injection well. The following illustrative examples demonstrate how the labelled gas can be used to assess, by analysis of produced fluids, the process and to control operating parameters accordingly.

In one example, the efficiency of gas transport from a reaction zone to the production well can be assessed by using labelled δ¹³C methane injected in the injection well to trace communication through the reservoir. If the efficiency of the gas transport is outside of an acceptable operating range, then the amount of gas phase in the injected fluids can be raised so that the gas saturation in the reservoir increases. Alternatively, the concentration of nutrient in the injected fluids can be increased to further stimulate microbes to produce more gas to raise the gas saturation in the reservoir. Alternatively, the injection period can be increased to allow more nutrient injection into the reservoir. Alternatively, water from a water zone could be produced from the reservoir to lower the pressure in the reservoir so that gas comes out of solution further raising the gas saturation.

In another example, the efficiency of hydrogen consumption relative to methane production (using δD on produced methane) can be assessed, by adding deuterium labelled hydrogen gas to the injected fluids and monitoring the deuterium content of the produced methane. If the efficiency of the hydrogen consumption is outside of an acceptable operating range, then gas production rate or soak time can be altered to more efficiently convert carbon dioxide to methane using hydrogen. Other data, such as microbiological analysis and chemical analysis of produced waters, can be used to define a remediation strategy.

In another example, the efficiency of carbon dioxide reduction to methane can be assessed by using injection of δ¹³C labelled CO₂ and monitoring the carbon isotopic composition of the carbon dioxide and methane content of produced gases. If the efficiency of the carbon dioxide reduction to methane is outside of an acceptable operating range, then gas production rate or soak time can be altered to more efficiently convert carbon dioxide to methane using hydrogen. Other data, such as microbiological analysis and chemical analysis of produced waters and oils, can be used to define a remediation strategy.

In another example, the extent of acetoclastic methanogenesis can be assessed by injecting carbon isotopically labeled acetate in the injected nutrient solution or injected waters and analyzing the carbon isotopic composition of produced carbon dioxide and methane. The level of acetoclastic methanogenic activity acting in the reservoir then can be adjusted in the isotope fractionation model, to provide more effective process control description and a new set of updated control parameter diagrams as in FIGS. 17 and 18, for example.

In some implementations, the production flow rate can be used to control the reservoir pressure during the internal when methanogenesis is occurring. To raise the reservoir pressure, the production flow rate can be reduced. To lower the reservoir pressure, the production flow rate can be raised. Together with the equation set forth above, that relates the gas saturation and reservoir pressure through the formation volume factors, a reservoir pressure can be determined that will result in a gas saturation that exceeds the critical gas saturation and provides gas mobility. Thus, to some extent, the production flow rate can be used to adjust the gas saturation in the reservoir, and there to control the mobility of the gas.

In some implementations, the reservoir pressure can be adjusted by moderating fluid production and injection rates into the reservoir, such that, in combination with changes in gas saturation related to microbial gas generation occurring in the reservoir, the reservoir is maintained at a gas saturation above the critical gas saturation and gas production can persist. Reservoir simulations of the injection and production process, constrained by gas, oil and water flow rates and pressures, together with produced gas and oil compositions to provide biogenerated gas rates, can be used to determine and control the fluid injection and gas and oil production rates to maintain the process at the desired state of biogenerated gas production.

After the microbes have been stimulated, if the reservoir pressure exceeds the initial reservoir pressure, this indicates the formation of biogenerated gas in the reservoir. After the reservoir goes on production, if the reservoir pressure still climbs, then the amount of biogenerated gas is greater than the amount of fluid being produced from the reservoir. If the pressure starts to fall, then the amount of fluid being removed from the reservoir is greater than the reservoir volume of gas generated by the microbes. Thus, the preferred condition is to try and achieve a relatively constant pressure by producing from the reservoir fluids at the same rate as gas is biogenerated in situ. Providing gas is being produced from the reservoir, which would require that the gas saturation is higher than the critical gas saturation, operating at a constant pressure can maintain the gas saturation above the critical gas saturation throughout production. To achieve higher gas production rates, e.g., so as to enhance the economics of the process, gas production can be raised so that the pressure in the reservoir falls as gas production continues. In this case, at some point where gas production became uneconomic, production can be shut-in and additional fluids (including modifiers, water, and gas) can be injected into the reservoir. An alternative is to shut-in the reservoir and allow the microbes to generate gas volume. In both cases, the reservoir pressure can be monitored to determine the amount of gas generated.

### Example System

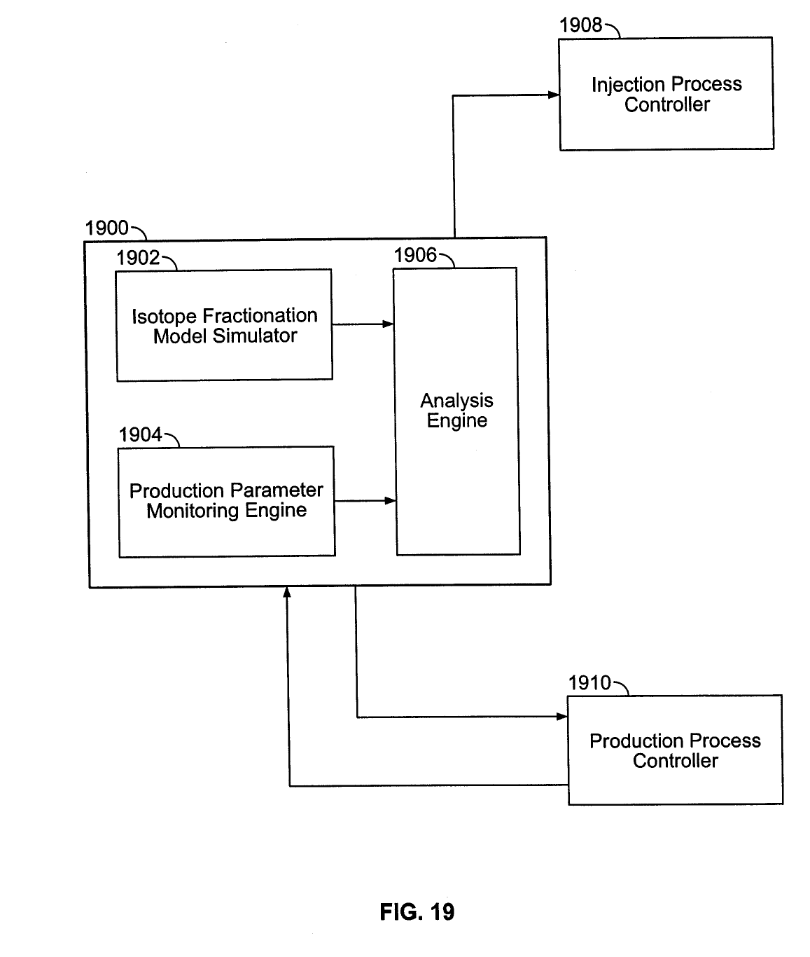

Referring now to FIG. 19, a schematic representation is shown of an example system 1900 that can be used to implement the methods for enhancing production of biogenerated gas from a zone as described herein. The system can include an isotope fractionation model simulator 1902 that can be used to predict the composition of produced gas when production of the biogenerated gas is occurring under optimal or non-optimal conditions. The predicted composition can include the bulk composition and the isotopic composition. The system includes a production parameter monitoring engine 1904 configured to receive production parameter data from the production process 1910. The production parameter data and information about predicted compositions of produced gas can be input into an analysis engine 1906 from the production parameter monitoring engine 1904 and the isotope fractionation model simulator 1902 respectively. The analysis engine 1906 can be configured to compare the actual production parameter to the predicted compositions and provide an analysis as to the efficacy of the current production process 1910.

In the example implementation shown, the analysis engine 1906 is configured to provide control data to the injection process 1908 and the production process 1910, such that adjustments to one or both of the injection and production processes can be made in response to the analysis provided by the analysis engine 1906. For example, the monitored production parameters can include reservoir pressure and an isotopic composition of at least one of either methane or carbon dioxide or hydrogen included in the produced gas. Based on a comparison of the monitored production parameters to the input from the isotope fractionation model simulator, the analysis engine 1906 can adjust at least one of stimulation of the microbial conversion (to control the composition of the produced gas) or gas saturation in the zone (to control mobility of the biogenerated methane) by adjusting the injection process 1908 and/or the production process 1910. In other implementations, the analysis engine 1906 provides an analysis, that can then be interpreted and applied to make adjustments by another entity, either automated or manually.

The system 1900 can be implemented using one or more computers, as is described further below. An engine, as the term is used throughout this application, can be a piece of hardware that encapsulates a function, can be firmware or can be a software application. An engine can perform one or more functions, and one piece of hardware, firmware or software can perform the functions of more than one of the engines described herein. Similarly, more than one piece of hardware, firmware and/or software can be used to perform the function of a single engine described herein.

In some implementations, the system 1900 can be implemented as software, firmware or hardware coupled (e.g., logically) with a reservoir simulation application to receive output from the reservoir simulation application, or to provide control data for history matching or other purposes to the reservoir simulation application. In some implementations, the system 1900 can be included as one or more subroutines in a coupled reservoir simulation application and/or process control software package. Other configurations are possible.

### Other Implementations

Modifying the Critical Gas Saturation in the Reservoir

The lower the critical gas saturation, the sooner the gas phase can become mobile. Accordingly, in some implementations, a modifier can be injected into the zone to lower the critical gas saturation. For example, a non-sulphate containing surfactant can be introduced into the reservoir along with the modifiers to stimulate gas mobilization. Example surfactants include polyethyleneglycol distearate, sodium lauroyl lactylate, fatty acid salts, and other agents that reduce the interfacial tension between gas and liquid.

In other implementations, the fluid injected into the zone can be warmed relative to the reservoir temperature prior to injection. Introducing a higher temperature fluid into the reservoir can increase the overall temperature in the zone and thereby decrease the critical gas saturation. Other techniques that can lower the critical gas saturation can also be used.

Production of Other Types of Gas

As was discussed above, the methods and systems described herein can be used to produce to surface other types of gas converted from petroleum, and methane is but one example. For example, known techniques for stimulating microbial hydrogen production, such as those described in PCT Application WO 2005/115648 entitled “Process for Stimulating Production of Hydrogen from Petroleum in Subterranean Formations”, filed by Larter, et al., can be used to convert petroleum to hydrogen. The methods and systems described herein to produce biogenerated gas to surface can be implemented to produce biogenerated hydrogen. That is, while producing the hydrogen gas to surface, pressure in the reservoir can be monitored as well as the composition of the produced gas, including monitoring an isotopic composition of at least one of either hydrogen, methane or carbon dioxide included in the produced gas. Monitoring of the activity of methanogens using microbiological analysis of produced waters also can be employed to identify if agents to restrict methanogenesis are required to be injected (e.g., bromoethanesulphonates). In one example, if sulphate reducing organisms, which can consume hydrogen, are found in produced waters, then sodium molybdate can be injected in the injection waters.

In some implementations, to enhance hydrogen production, effective removal of hydrogen from the site of generation can be accomplished using an injected sparge gas, such as nitrogen, carbon dioxide or methane, or microbial generation of large enough quantities of microbially generated gases (CO₂ or CH₄) in other zones of the reservoir. Typically, for thermodynamic reasons, the production of hydrogen occurs at low net hydrogen concentrations in the produced gases, and laboratory studies of hydrogen production using microbiological samples for the reservoir of interest can be used to assess the critical hydrogen concentrations, at which hydrogen production ceases or slows. These values can then be used as control variables in a hydrogen production process whereby reservoir pressure and hydrogen concentrations in produced gases are assessed and used to decide if more sparge gas injection is needed to lower net hydrogen concentrations in the hydrogen generation zone. Injection of small amounts of deuterium labeled gas to the generation zone can be used to assess, by hydrogen isotopic analysis of produced methane or hydrogen, if methanogenesis is persisting and needs to be controlled.

Based on the reservoir pressure and the composition of the produced gas, the reservoir pressure can be controlled to increase the duration of the gas saturation in the reservoir exceeding the critical gas saturation to enhance the production of biogenerated hydrogen from the reservoir. Biogenerated hydrogen and methane are but two examples. Enhancing production to surface of other biogenerated gases, including for example carbon dioxide, can also be achieved using the methods and systems described herein.

Conversion of Petroleum to Biogenerated Gas and Production from a Surface Reservoir

The above discussion described implementations of the methods and systems described wherein the reservoir is a subterranean reservoir. However, in other implementations, petroleum and other material present in a subterranean reservoir can be mined and placed within a reservoir that may be partially or entirely above surface, for example, a man made reservoir. The same methods and systems described to stimulate the conversion of the petroleum into a biogenerated gas (e.g., methane) and to produce the biogenerated gas from the reservoir can be used, whether or not the petroleum is in an original subterranean reservoir or has been moved to a different location, e.g., a manmade reservoir or petroleum contaminated site.

Computer Implemented Controls

Techniques are described herein for controlling the reservoir pressure to increase the duration of the gas saturation exceeding the critical gas saturation to enhance the production of biogenerated gas from the zone. That is, adjustments can be made to the stimulation of microbial conversion and/or the gas saturation. In some implementations, computer software can be used to automatically determine what adjustments can be made to the injection and/or production processes so as to enhance the production of biogenerated gas from the zone. For example, as described above in reference to FIG. 19, one or more components of the system 1900 can be implemented, at least in part, with computer software. Monitored production parameters, e.g., reservoir pressure measurements, gas production flow rate data, and composition analyses of the produced gas, can be input into the computer software. The input data can be used to compare the operating process to optimal operating conditions (as was described above). If deviations from optimal operating conditions are detected, the computer software can determine, depending on the particular deviation, adjustments to one or more operating parameters to correct the operating conditions.

In some implementations, the computer software generates a report identifying the recommended adjustments to the operating parameters. In other implementations, computer software can also be used to automatically implement the recommendations. For example, if the computer software recommends reducing the injector flow rate, a signal can be automatically sent to a controller at the injector to thereby reduce the injector flow rate. In other modes, control algorithms such as proportional-integral-derivative control can be used to set the opening on the control valve to control the reservoir pressure as measured at the base of the production well. Any form of feedback or feed forward control algorithm can be used to monitor and control the system so the pressure set-point at one or more of the measured pressure points, for example, in each of the injection and production wells, can be maintained.

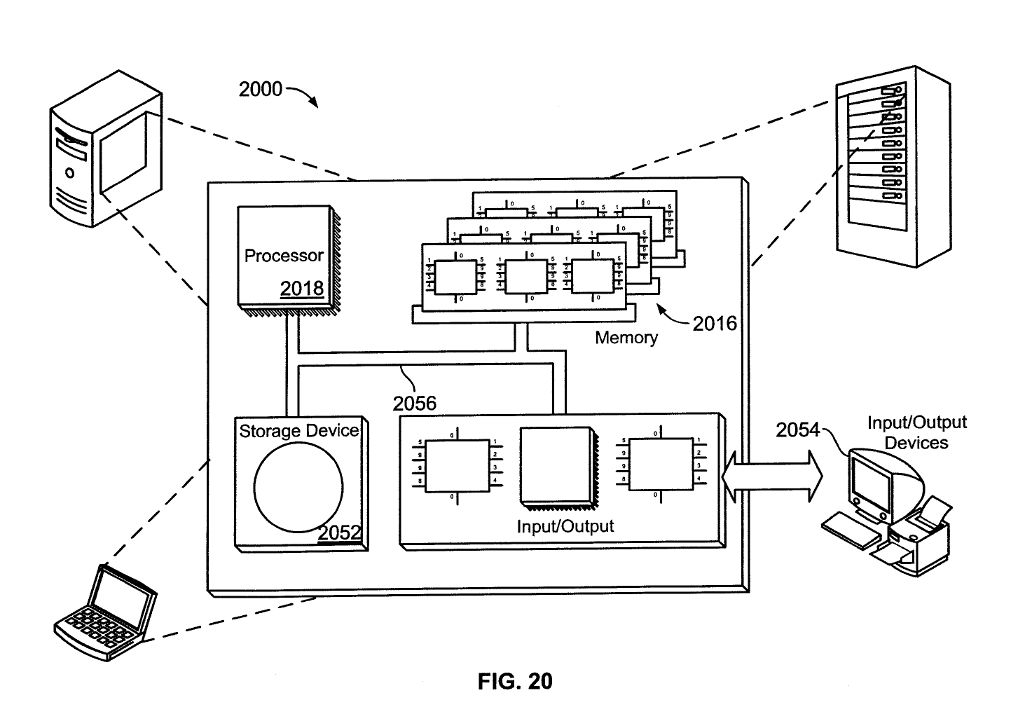

FIG. 20 is a schematic diagram of an example computer system 2000. The system 2000 can be used for performing actions and methods described above. The system 2000 can include a processor 2018, a memory 2016, a storage device 2052, and input/output devices 2054. Each of the components 2018, 2016, 2052, and 2054 can be interconnected using a system bus 2056. The processor 2018 is capable of processing instructions within the system 2000. These instructions can implement one or more aspects of the systems, components, and techniques described above. In some implementations, the processor 2018 is a single-threaded processor. In other implementations, the processor 2018 is a multi-threaded processor. The processor 2018 can include multiple processing cores and is capable of processing instructions stored in the memory 2016 or on the storage device 2052 to display graphical information for a user interface on the input/output device 2054.

The memory 2016 is a computer readable medium such as volatile or non-volatile that stores information within the system 2000. The memory 2016 can store processes related to the functionality of network routing, for example. The storage device 2052 is capable of providing persistent storage for the system 2000. The storage device 2052 can include a floppy disk device, a hard disk device, an optical disk device, or a tape device, or other suitable persistent storage mediums. The storage device 2052 can store the various databases described above. The input/output device 2054 provides input/output operations for the system 2000. The input/output device 2054 can include a keyboard, a pointing device, and a display unit for displaying graphical user interfaces.

The computer system shown in FIG. 20 is but one example. In general, embodiments of the subject matter and the functional operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer program products, i.e., one or more modules of computer program instructions encoded on a tangible program carrier for execution by, or to control the operation of, data processing apparatus. The tangible program carrier can be a propagated signal or a computer readable medium. The propagated signal is an artificially generated signal, e.g., a machine generated electrical, optical, or electromagnetic signal, that is generated to encode information for transmission to suitable receiver apparatus for execution by a computer. The computer readable medium can be a machine readable storage device, a machine readable storage substrate, a memory device, a composition of matter effecting a machine readable propagated signal, or a combination of one or more of them.

The term “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them. A computer program (also known as a program, software, software application, script, or code) can be written in any form of programming language, including compiled or interpreted languages, or declarative or procedural languages, and it can be deployed in any form, including as a stand alone program or as a module, component, subroutine, or other unit suitable for use in a computing environment.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

A number of embodiments of the invention have been described. Nevertheless, it will be understood that various modifications may be made without departing from the spirit and scope of the invention. Accordingly, other embodiments are within the scope of the following claims.

1. A method for producing biogenerated gas from a zone in a reservoir, comprising:
stimulating microbial conversion of petroleum in the zone to biogenerated methane;
producing gas including biogenerated methane from the zone to surface;
while producing the gas, monitoring production parameters including reservoir pressure and an isotopic composition of at least one of either methane or carbon dioxide or hydrogen included in the produced gas; and
based on the monitored production parameters, adjusting at least one of stimulation of the microbial conversion to control the composition of the produced gas or gas saturation in the zone to control mobility of the biogenerated methane.

2. The method of claim 1, wherein monitoring production parameters further comprises monitoring a flow rate of the produced gas.

3. The method of claim 1, wherein monitoring production parameters further comprises monitoring a chemical composition of the produced gas.

4. The method of claim 3, wherein monitoring the chemical composition of the produced gas comprises monitoring the amounts of methane, carbon dioxide and hydrogen included in the produced gas.

5. The method of claim 1, wherein monitoring an isotopic composition comprises monitoring a carbon isotopic composition of at least one of either methane or carbon dioxide included in the produced gas.

6. The method of claim 1, wherein monitoring an isotopic composition comprises monitoring a hydrogen isotopic composition of methane or hydrogen included in the produced gas.

7. The method of claim 1, wherein monitoring an isotopic composition comprises monitoring an oxygen isotopic composition of carbon dioxide included in the produced gas.

8. The method of claim 1, wherein monitoring production parameters further comprises monitoring at least one of either hydrogen or oxygen isotopic composition of produced water.

9. The method of claim 1, further comprising:
before producing gas from the zone, monitoring an increase in reservoir pressure in the zone, and based on the reservoir pressure determining the zone's gas saturation;
commencing production of gas from the zone when the zone's gas saturation reaches a threshold gas saturation, where the threshold gas saturation is based on a critical gas saturation of the zone.

10. The method of claim 9, wherein the threshold gas saturation is in the range of approximately 85% to 95% of the critical gas saturation.

11. The method of claim 9, wherein the threshold gas saturation is the critical gas saturation.

12. The method of claim 1, wherein stimulating microbial conversion of petroleum in the zone to biogenerated methane comprises injecting one or more modifiers into the reservoir to stimulate microbial conversion of the petroleum to methane.

13. The method of claim 12, further comprising:
injecting fluid into the reservoir, the fluid including gas and water.

14. The method of claim 13, wherein adjusting at least one of stimulation of the microbial conversion to control the composition of the produced gas or gas saturation in the zone to control mobility of the biogenerated methane includes:
adjusting an injector well flow rate for injecting at least one of either the one or more modifiers or the fluid into the reservoir.

15. The method of claim 13, wherein adjusting at least one of stimulation of the microbial conversion to control the composition of the produced gas or gas saturation in the zone to control mobility of the biogenerated methane includes:
adjusting a production well flow rate.

16. The method of claim 13, wherein adjusting at least one of stimulation of the microbial conversion to control the composition of the produced gas or gas saturation in the zone to control mobility of the biogenerated methane includes:
adjusting a composition of the one or more modifiers injected into the reservoir.

17. The method of claim 13, wherein adjusting at least one of stimulation of the microbial conversion to control the composition of the produced gas or gas saturation in the zone to control mobility of the biogenerated methane includes:
adjusting a quantity of the one or more modifiers injected into the reservoir.

18. The method of claim 13, wherein adjusting at least one of stimulation of the microbial conversion to control the composition of the produced gas or gas saturation in the zone to control mobility of the biogenerated methane includes:
adjusting a composition of the one or more modifiers injected into the reservoir.

19. The method of claim 13, wherein adjusting at least one of stimulation of the microbial conversion to control the composition of the produced gas or gas saturation in the zone to control mobility of the biogenerated methane includes:
adjusting a composition of the gas included in the fluid injected into the reservoir.

20. The method of claim 13, wherein adjusting at least one of stimulation of the microbial conversion to control the composition of the produced gas or gas saturation in the zone to control mobility of the biogenerated methane includes:
adjusting a quantity of the gas included in the fluid injected into the reservoir.

21. The method of claim 13, wherein adjusting at least one of stimulation of the microbial conversion to control the composition of the produced gas or gas saturation in the zone to control mobility of the biogenerated methane includes:
adjusting a quantity of the water included in the fluid injected into the reservoir.

22. The method of claim 13, wherein adjusting at least one of stimulation of the microbial conversion to control the composition of the produced gas or gas saturation in the zone to control mobility of the biogenerated methane includes:
adjusting a duration of injection, soak and production cycles for the reservoir.

23. The method of claim 13, wherein adjusting at least one of stimulation of the microbial conversion to control the composition of the produced gas or gas saturation in the zone to control mobility of the biogenerated methane includes:
ceasing production of the gas from one or more wells and commencing production of the gas from one or more different wells.

24. The method of claim 13, wherein:
injecting fluid into the zone comprises injecting fluid into the zone through a first set of one or more wells; and
producing gas from the zone comprises producing gas from the zone through a second set of one or more wells.

25. The method of claim 24, wherein:
injecting fluid into the zone is concurrent with producing gas from the zone.

26. The method of claim 24, wherein:
injecting fluid into the zone ceases while producing gas from the zone.

27. The method of claim 13, wherein injecting fluid into the zone comprises injecting fluid into the zone through a first set of one or more wells and producing gas from the zone comprises producing gas from the zone through a second set of one or more wells, the method further comprising:
allowing a soak cycle to endure in a region of the zone situated proximate a third set of one or more wells, while injection and production occur from the first and second set of one or more wells; and
during a later cycle, allowing the first set of one or more wells to endure a soak cycle, injecting fluid into the zone through the second set of one or more wells and producing gas from the zone from the third set of one or more wells.

28. The method of claim 1, further comprising:
using an isotope fractionation model to predict the isotopic and bulk gas composition of the produced gas when gas production is occurring under optimal conditions;
comparing the actual isotopic and bulk gas composition of the produced gas to the predicted isotopic and bulk composition; and
using the comparison to adjust at least one of stimulation of the microbial conversion to control the composition of the produced gas or gas saturation in the zone to control mobility of the biogenerated methane.

29. A method for producing biogenerated methane from a zone in a reservoir that can support biodegradation of petroleum to produce biogenerated methane by methanogenesis, comprising:
injecting one or more modifiers into the reservoir to stimulate microbial conversion of the petroleum to biogenerated methane;
injecting fluid into the reservoir, the fluid including gas and water;
while gas saturation in the zone exceeds a critical gas saturation of the zone, producing gas from the zone to surface including producing the biogenerated methane;
while producing the gas, monitoring production parameters including the pressure in the reservoir, gas production flow rate and composition of the produced gas, including monitoring an isotopic composition of at least one of either methane or carbon dioxide or hydrogen included in the produced gas; and
based on the monitored production parameters, controlling injection into and/or production from the zone to enhance production of biogenerated methane from the zone.

30. The method of claim 29, wherein controlling injection into and/or production from the zone to enhance production of biogenerated methane from the zone comprises:
controlling an injector well flow rate.

31. The method of claim 29, wherein controlling injection into and/or production from the zone to enhance production of biogenerated methane from the zone comprises:
controlling a production well flow rate.

32. The method of claim 29, wherein controlling injection into and/or production from the zone to enhance production of biogenerated methane from the zone comprises:
controlling a composition of the one or more modifiers injected into the reservoir.

33. The method of claim 29, wherein controlling injection into and/or production from the zone to enhance production of biogenerated methane from the zone comprises:
controlling a quantity of the one or more modifiers injected into the reservoir.

34. The method of claim 29, wherein controlling injection into and/or production from the zone to enhance production of biogenerated methane from the zone comprises:
controlling a composition of the gas injected into the reservoir.

35. The method of claim 29, wherein controlling injection into and/or production from the zone to enhance production of biogenerated methane from the zone comprises:
controlling a quantity of the gas injected into the reservoir.

36. The method of claim 29, wherein controlling injection into and/or production from the zone to enhance production of biogenerated methane from the zone comprises:
controlling a duration of injection, soak and production cycles for the reservoir.

37. The method of claim 29, further comprising:
before producing gas from the zone, monitoring a change in reservoir pressure in the zone in response to an increase in biogenerated methane and the injected fluid;
based on the reservoir pressure, determining the zone's gas saturation; and
commencing production of gas from the zone when the zone's gas saturation reaches a threshold gas saturation, where the threshold gas saturation is based on the critical gas saturation.

38. The method of claim 37, wherein the threshold gas saturation is in the range of approximately 85% to 95% of the critical gas saturation.

39. The method of claim 37, wherein the threshold gas saturation is the critical gas saturation.

40. The method of claim 1, further comprising:
using an isotope fractionation model to predict the composition of the produced gas when gas production is occurring under optimal conditions;
comparing the actual composition of the produced gas to the predicted composition; and
using the comparison to control injection into and/or production from the zone to enhance production of biogenerated methane from the zone.

41. A method for producing biogenerated gas from a zone in a reservoir, comprising:
stimulating microbial conversion of petroleum in the zone to biogenerated gas;
producing gas including the biogenerated gas from the zone to surface;
while producing the gas, monitoring production parameters including reservoir pressure and an isotopic composition of the produced gas; and
based on the monitored production parameters, adjusting at least one of stimulation of the microbial conversion to control the composition of the produced gas or gas saturation in the zone to control mobility of the biogenerated gas.

42. The method of claim 41, wherein monitoring the production parameters further comprises monitoring a bulk gas composition of the produced gas.

43. The method of claim 41, wherein the biogenerated gas is hydrogen.

44. The method of claim 41, wherein the biogenerated gas is carbon dioxide.

45. The method of claim 41, wherein the biogenerated gas is methane.

46. A system for producing biogenerated gas from a zone in a reservoir, the system comprising:
an isotope fractionation model simulator configured to predict a composition of produced gas from the zone when production of biogenerated gas is occurring under optimal conditions;
a production parameter monitoring engine configured to receive production parameter data about a production process, the production parameter data including reservoir pressure and an isotopic composition of at least one or either methane or carbon dioxide or hydrogen included in the produced gas;
an analysis engine configured to:
receive the predicted compositions of produced gas from the isotope fractional model simulator and the production parameter data from the production parameter monitoring engine;
compare the predicted composition of produced gas to the actual isotopic composition of the produced gas; and
provide an analysis of the efficacy of the production process based on the comparison.

47. The system of claim 46, wherein the composition predicted by the isotope fractionation model simulator include a bulk gas composition and an isotopic composition.

48. The system of claim 46, wherein the analysis engine is further configured to provide control signals to a controller for the production process to adjust the production process in response to the analysis.

49. The system of claim 48, wherein the analysis engine is further configured to provide control signals to a controller for an injection process injecting at least one of modifiers to stimulate microbial conversion of petroleum to biogenerated gas or fluid including gas and water into the reservoir, where the control signals adjust the injection process in response to the analysis.
